(19) United States
(12) Patent Application Publication
(10) Pub. No.: US 2025/0032485 A1
(43) Pub. Date: Jan. 30, 2025

(54) APPLICATION OF IMIDAZO [1,2-A] PYRAZINE COMPOUNDS OR THEIR PHARMACEUTICALLY ACCEPTABLE SALTS IN THE PREPARATION OF ANTI-CORONAVIRUS DRUGS

(71) Applicant: GUANGZHOU MEDICAL UNIVERSITY, Guangzhou (CN)

(72) Inventors: XIYONG YU, Guangzhou (CN); AO SHEN, Guangzhou City (CN); LIXIN ZHAO, Guangzhou (CN); HUA TAO, Guangzhou (CN); NANSHAN ZHONG, Guangzhou (CN)

(21) Appl. No.: 18/770,569

(22) Filed: Jul. 11, 2024

(30) Foreign Application Priority Data
Jul. 14, 2023 (CN) 202310867035.4

(51) Int. Cl. A61K 31/4985; A61P 31/14

(52) U.S. Cl. CPC A61K 31/4985; A61P 31/14

(57) ABSTRACT

The present disclosure provides methods, compounds and compositions that are relevant to DNA-repair and DNA-repair proteins such as tyrosyl-DNA phosphodiesterase 1 (TDP1) and type I topoisomerase (TOP1).

### CROSS-REFERENCE TO RELATED APPLICATIONS

This application claims to the benefit of priority from Chinese Application No. 202310867035.4 with a filing date of Jul. 14, 2023. The content of the aforementioned applications, including any intervening amendments thereto, are incorporated herein by reference.

### TECHNICAL FIELD

The present disclosure relates to the technical field of pharmaceutical technology, in particular to a new application of imidazo [1, 2-a] pyrazine compounds or their pharmaceutically acceptable salts in the preparation of anti-coronavirus drugs.

### BACKGROUND

Coronavirus is a large class of viruses widely present in nature. Currently, it is known that seven types of coronaviruses can infect humans, belonging to the human coronavirus. After infecting hosts with different coronaviruses, they all undergo the same life cycle: the virus genes first translate and express two polyproteins, pp1a and pp1ab, which need to be cleaved into independent non-structural proteins (Nsp1 to Nsp16) by two types of coronavirus proteases: papain-like protease (PLpro) and 3C-like protease (3CLpro) before virus replication can be achieved. The PLpro of SARS-CoV-2 and other human coronaviruses exhibit high homology and share same function, but they have low similarity with human proteases. In view of the important role of PLpro in viral replication and its high conservatism in coronavirus, it is an ideal target for the development of anti-coronavirus drugs. For PLpro in SARS-CoV-2, new structural small molecule inhibitors can be obtained through directly screening, which will play an important role in the treatment of coronavirus infection, especially SARS-CoV-2 infection.

### SUMMARY

Based on this, the present disclosure has discovered a class of imidazo [1, 2-a] pyrazine compounds, which have good inhibitory activity against coronavirus PLpro and can be used to prepare drugs against coronavirus.

The present disclosure includes the following technical solutions.

An application of imidazo [1, 2-a] pyrazine compounds or their pharmaceutically acceptable salts in a preparation of anti-coronavirus drugs, wherein the imidazo [1, 2-a] pyrazine compounds of the present disclosure are selected from the following compounds:

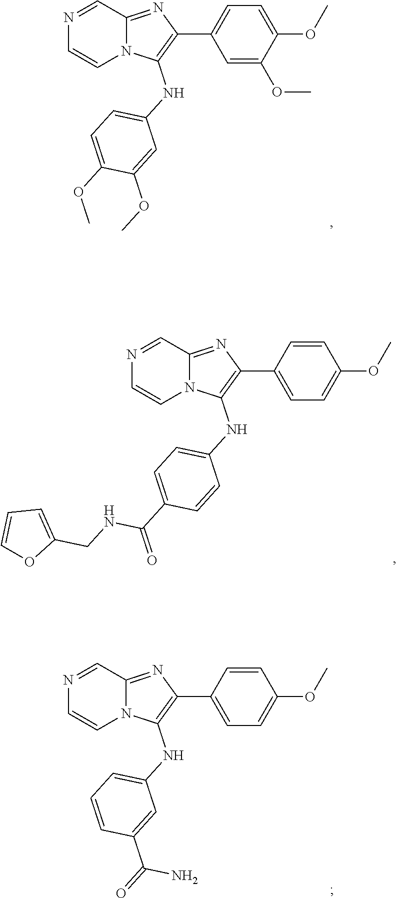 [SMILES: COc1ccc(Nc2c(-c3ccc(OC)c(OC)c3)nc3cnccn23)cc1OC ; COc1ccc(-c2nc3cnccn3c2Nc2ccc(C(=O)NCc3ccco3)cc2)cc1 ; COc1ccc(-c2nc3cnccn3c2Nc2cccc(C(N)=O)c2)cc1]

and

The coronavirus is SARS-CoV-2.

Compared with the prior art, the present disclosure finds that a class of derivative compounds with imidazo [1, 2-a] pyrazine as the core structure can effectively inhibit the PLpro activity of SARS-CoV-2. The structure of these compounds is obviously different from any known inhibitor of coronavirus PLpro. They are a class of new small molecule inhibitors that can inhibit the activity of coronavirus PLpro, and have the advantages of good druggability and low cytotoxicity. The preferred compounds have no obvious cytotoxicity to BEAS-2B cells within the tested concentration range (up to 200 μM). Therefore, the imidazo [1, 2-a] pyrazine derivative compounds discovered in the present disclosure have the effect of anti-coronavirus infection, especially anti-SARS-CoV-2 infection, and are expected to become broad-spectrum anti-coronavirus drugs.

### BRIEF DESCRIPTION OF THE DRAWINGS

FIG. 1 shows the analysis results of the druggability of compound P19453.

FIG. 2 shows the analysis results of the druggability of compound P22445.

FIG. 3 shows the analysis results of the druggability of compound P22475.

FIG. 4 shows the cytotoxicity results of compounds P19453, P22445, and P22475.

### DETAILED DESCRIPTION OF THE EMBODIMENTS

The following will further illustrate the technical solution of the present disclosure through specific embodiments.

### Embodiment 1: Test of the Inhibitory of Imidazo [1, 2-a] Pyrazine Compounds on PLpro Activity of SARS-CoV-2

The inhibitory effect of imidazo [1, 2-a] pyrazine derivative compounds on PLpro activity of SARS-CoV-2 was tested by an enzyme activity test in vitro. PLpro can hydrolyze substrate RLRGG-AMC to generate free AMC. The fluorescence emission value at a wavelength of 460 nm of free AMC excited at a wavelength of 380 nm was detected by a microplate reader to reflect PLpro activity. After adding small molecule inhibitors, PLpro activity is inhibited and AMC fluorescence emission value decreases. After adding different small molecule compounds with a fixed concentration (50 μM) to the reaction system, the changes in PLpro activity were reflected by the reading of the microplate reader, and their inhibition rate on PLpro activity was calculated. The specific steps are as follows:
1.1 The composition of the reaction buffer is DTT 10 mM, Tris 20 mM, NaCl 150 mM, 2% DMSO. The substrate RLRGG-AMC purchased from Beyotime was dissolved in DMSO to make a 10 mM stock solution. The PLpro of SARS-CoV-2 purchased from Sino Biological Inc. was dissolved to make a 125 ng/μL stock solution with nuclease-free water.
1.2 Preparation of substrate: adding 2 μL of substrate stock solution to 198 μL reaction buffer, and mixing well to obtain 100 μM of substrate working solution.
1.3 Preparation of PLpro: adding 10 μL of PLpro stock solution to 90 μL reaction buffer, and mixing well to obtain 12.5 ng/μL of PLpro working solution.
1.4 For the experimental group, mixing 10 μL of PLpro working solution with 2 μL of imidazo [1, 2-a] pyrazine compounds as shown in Table 1 (all purchased from Shanghai Topscience Co., Ltd.) (final concentration 50 μM); for the control group, not adding any compounds, but mixing 10 μL of PLpro working solution with 2 μL of compound solvent (DMSO); then incubate at 37° C. for 10 minutes.
1.5 quickly adding 10 μL of the substrate working solution prepared in 1.2 to the 1.4 system, then using a microplate reader to continuously read for 15 minutes under 37° C. incubation conditions, with a 30 second interval between each cycle, wherein the excitation wavelength is set to 380 nm and the emission wavelength is set to 460 nm.
1.6 Calculation of inhibition rate: using time min as the X-axis and fluorescence value RFU as the Y-axis, fitting the linear region to obtain RFU/min as enzyme activity, then calculating the inhibition rate of the compound on PLpro based on the difference in enzyme activity between the experimental group and the control group. The calculation formula is as follows: inhibition rate=(1−enzyme activity of experimental group/enzyme activity of control group)×100%. The results of testing of enzyme activity in vitro are shown in Table 1. The results showed that 168 compounds based on the core structure of imidazo [1, 2-a] pyrazine have different degrees of inhibition on PLpro, and 21 compounds have more than 45% inhibition rate on PLpro of SARS-CoV-2 at 50 μM, which was used for subsequent IC50 determination.

TABLE 1
Inhibition effect of imidazo [1,2-a] pyrazine compounds on
coronavirus PLpro activity at a concentration of 50 μM

| | Inhibition | | |
|---|---|---|---|
| | rate at | | |
| Serial | 50 μM | | |
| number | (%) | IUPAC nomenclature | Structural formula |
| P22423 | 54.48 | 4-[[2-(4-methylphenyl) imidazo[1,2-a] pyrazin-3-yl]amino]- N-(2-morpholin-4- ylethyl)benzamide | 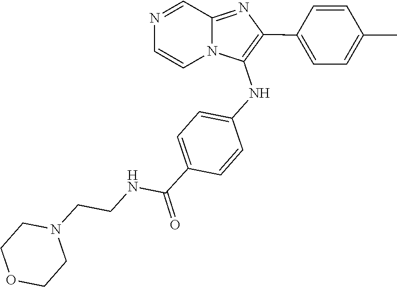 [SMILES: Cc1ccc(-c2nc3cnccn3c2Nc2ccc(C(=O)NCCN3CCOCC3)cc2)cc1] |
| P22471 | 52.83 | Nethyl-4-[[2-(4- methoxyphenyl) imidazo[1,2-a]pyrazin- 3-yl]amino]benzamide | 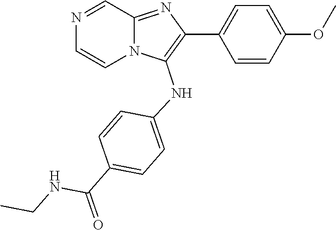 [SMILES: CCNC(=O)c1ccc(Nc2c(-c3ccc(OC)cc3)nc3cnccn23)cc1] |
| P22479 | 58.84 | N-(furan-2-ylmethyl)- 3-(4-methylsulfanyl- phenyl)imidazo [1,2-a]pyrazin-3-yl] amino]benzamide | 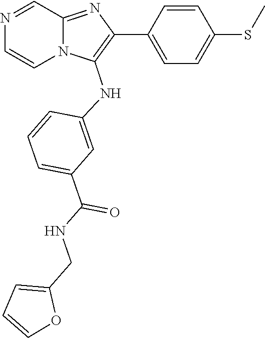 [SMILES: CSc1ccc(-c2nc3cnccn3c2Nc2cccc(C(=O)NCc3ccco3)c2)cc1] |
| P141 | 63.75 | methyl 3-[[2-(4-morpholin- 4-ylphenyl)imidazo [1,2-a]pyrazin-3- yl]amino]benzoate | 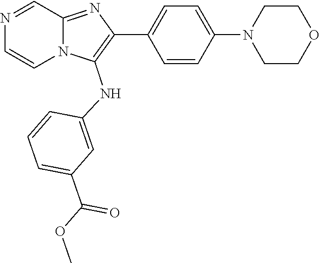 [SMILES: COC(=O)c1cccc(Nc2c(-c3ccc(N4CCOCC4)cc3)nc3cnccn23)c1] |
| P18224 | 54.57 | 4-[[2-(4-chlorophenyl) imidazo[1,2-a] pyrazin-3-yl]amino] benzoic acid | 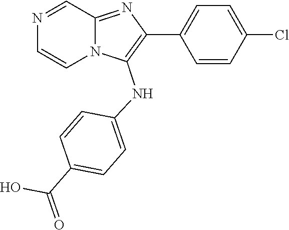 [SMILES: O=C(O)c1ccc(Nc2c(-c3ccc(Cl)cc3)nc3cnccn23)cc1] |
| P18234 | 20.48 | 4-[[2-(3-bromophenyl) imidazo[1,2-a] pyrazin-3-yl]amino] benzoic acid | 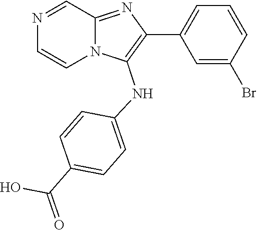 [SMILES: O=C(O)c1ccc(Nc2c(-c3cccc(Br)c3)nc3cnccn23)cc1] |
| P19442 | 61.12 | 4-[[2-(4- methoxyphenyl) imidazo[1,2-a] pyrazin-3-yl]amino] benzamide | 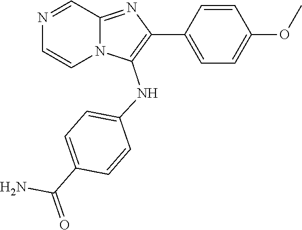 [SMILES: COc1ccc(-c2nc3cnccn3c2Nc2ccc(C(N)=O)cc2)cc1] |
| P19443 | 53.01 | 3-[[2-[4-(dimethyl- amino)phenyl]imidazo [1,2-a]pyrazin-3- yl]amino]-N-methyl- benzamide | 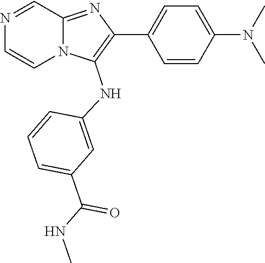 [SMILES: CNC(=O)c1cccc(Nc2c(-c3ccc(N(C)C)cc3)nc3cnccn23)c1] |
| P19444 | 42.19 | N-(1,3-benzodioxol- 5-yl)-2-(3,4-dimethoxy- phenyl)imidazo [1,2-a]pyrazin-3- amine | 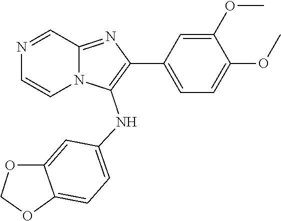 [SMILES: COc1ccc(-c2nc3cnccn3c2Nc2ccc3c(c2)OCO3)cc1OC] |
| P19445 | 50.21 | 4-[[2-(4-methyl- sulfanylphenyl)imidazo [1,2-a]pyrazin-3- yl]amino]benzoic acid | 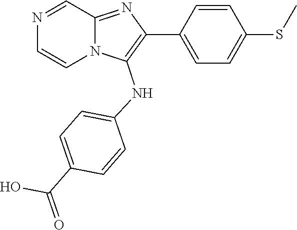 [SMILES: CSc1ccc(-c2nc3cnccn3c2Nc2ccc(C(=O)O)cc2)cc1] |
| P19446 | 65.76 | methyl 3-[[2-[4-(dimethyl- amino)phenyl]imidazo [1,2-a]pyrazin-3- yl]amino]benzoate | 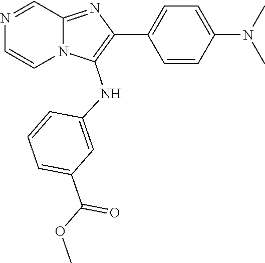 [SMILES: COC(=O)c1cccc(Nc2c(-c3ccc(N(C)C)cc3)nc3cnccn23)c1] |
| P19447 | 42.13 | 3-[[2-[4-(dimethyl- amino)phenyl]imidazo [1,2-a]pyrazin-3- yl]amino]benzoic acid | 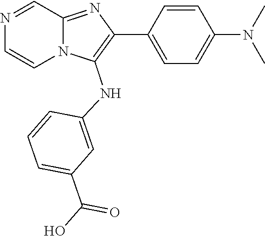 [SMILES: CN(C)c1ccc(-c2nc3cnccn3c2Nc2cccc(C(=O)O)c2)cc1] |
| P19452 | 53.11 | 4-[[2-(4-methyl- phenyl)imidazo[1,2-a] pyrazin-3-yl]amino] benzamide | 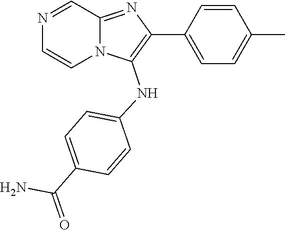 [SMILES: Cc1ccc(-c2nc3cnccn3c2Nc2ccc(C(N)=O)cc2)cc1] |
| P19453 | 65.52 | N,2-bis(3,4-dimethoxy- phenyl)imidazo [1,2-a]pyrazin-3- amine | 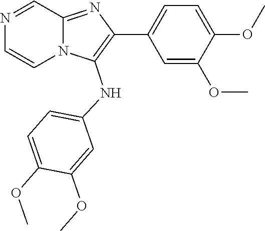 [SMILES: COc1ccc(Nc2c(-c3ccc(OC)c(OC)c3)nc3cnccn23)cc1OC] |
| P19454 | 53.82 | 4-[[2-(3,4-dimethoxy- phenyl)imidazo [1,2-a]pyrazin-3-yl] amino]benzamide | 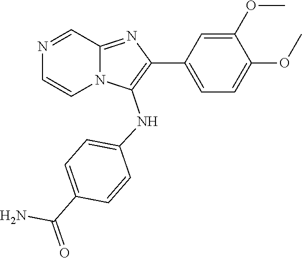 [SMILES: COc1ccc(-c2nc3cnccn3c2Nc2ccc(C(N)=O)cc2)cc1OC] |
| P19455 | 53.10 | 4-[[2-(4-hydroxy-3- methoxyphenyl) imidazo[1,2-a]pyrazin- 3-yl]amino]benzoic acid | 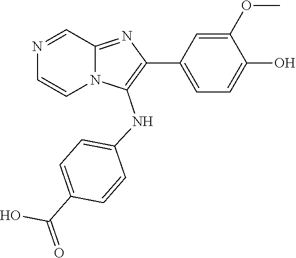 [SMILES: COc1cc(-c2nc3cnccn3c2Nc2ccc(C(=O)O)cc2)ccc1O] |
| P19456 | 47.83 | methyl 3-[[2-(3-chlorophenyl) imidazo[1,2-a] pyrazin-3-yl]amino] benzoate | 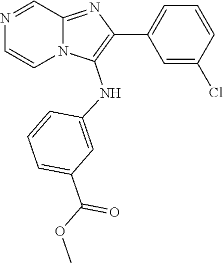 [SMILES: COC(=O)c1cccc(Nc2c(-c3cccc(Cl)c3)nc3cnccn23)c1] |
| P19457 | 39.69 | 3-[[2-(3-chlorophenyl) imidazo[1,2-a] pyrazin-3-yl]amino] benzoic acid | 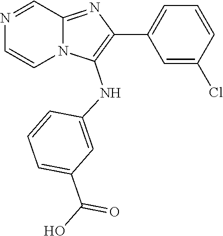 [SMILES: O=C(O)c1cccc(Nc2c(-c3cccc(Cl)c3)nc3cnccn23)c1] |
| P19462 | 64.99 | 4-[[2-(4-methyl- sulfanylphenyl)imidazo [1,2-a]pyrazin-3- yl]amino]benzamide | 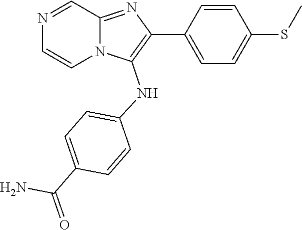 [SMILES: CSc1ccc(-c2nc3cnccn3c2Nc2ccc(C(N)=O)cc2)cc1] |
| P19463 | 40.74 | 2-(3,4-dimethoxy- phenyl)-N-(2-methyl phenyl)imidazo[1,2- a]pyrazin-3-amine | 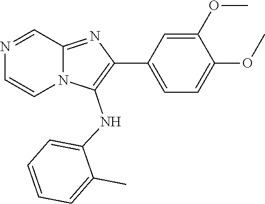 [SMILES: COc1ccc(-c2nc3cnccn3c2Nc2ccccc2C)cc1OC] |
| P19464 | 42.23 | 4-[[2-(3,4-dimethoxy- phenyl)imidazo[1,2- a]pyrazin-3-yl] amino]-N-methyl- benzamide | 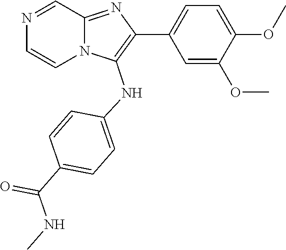 [SMILES: CNC(=O)c1ccc(Nc2c(-c3ccc(OC)c(OC)c3)nc3cnccn23)cc1] |
| P19465 | 15.92 | 4-[[2-(4-methyl- phenyl)imidazo[1,2-a] pyrazin-3-yl]amino] benzoic acid | 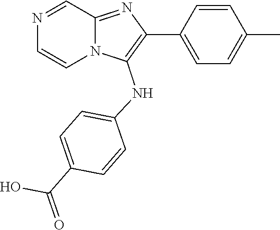 [SMILES: Cc1ccc(-c2nc3cnccn3c2Nc2ccc(C(=O)O)cc2)cc1] |
| P19466 | 31.90 | methyl 3-[[2-(5-bromo-2- hydroxyphenyl)imidazo [1,2-a]pyrazin-3- yl]amino]benzoate | 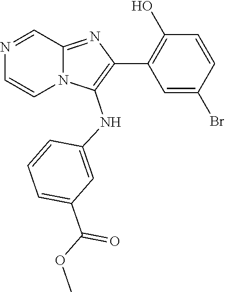 [SMILES: COC(=O)c1cccc(Nc2c(-c3cc(Br)ccc3O)nc3cnccn23)c1] |
| P19467 | 27.31 | 3-[[2-(3-bromophenyl) imidazo[1,2-a] pyrazin-3-yl]amino] benzoic acid | 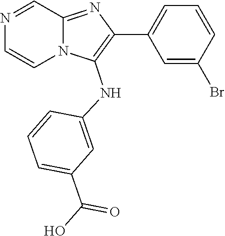 [SMILES: O=C(O)c1cccc(Nc2c(-c3cccc(Br)c3)nc3cnccn23)c1] |
| P19472 | 28.08 | 4-[[2-(4-methoxy- phenyl)imidazo[1,2-a] pyrazin-3-yl]amino]- N-methylbenzamide | 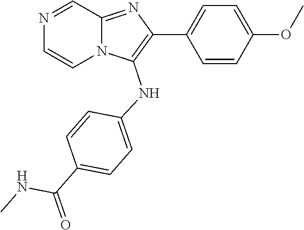 |
| P19473 | 37.48 | 2-(3,4-dimethoxy- phenyl)-N-(4-propan- 2-ylphenyl)imidazo [1,2-a]pyrazin-3- amine | 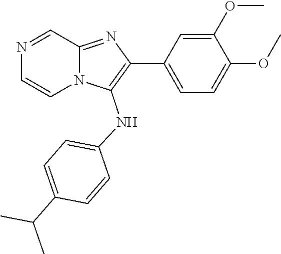 [SMILES: COc1ccc(-c2nc3cnccn3c2Nc2ccc(C(C)C)cc2)cc1OC] |
| P19474 | 31.65 | methyl 4-[3-(2-methylanilino) imidazo[1,2-a] pyrazin-2-yl]benzoate | 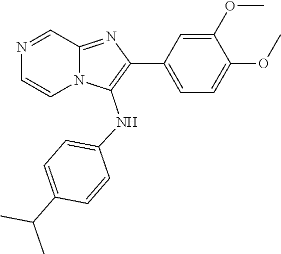 [SMILES: COc1ccc(-c2nc3cnccn3c2Nc2ccc(C(C)C)cc2)cc1OC] |
| P19475 | 35.13 | 4-[[2-[4-(dimethyl- amino)phenyl]imidazo [1,2-a]pyrazin-3- yl]amino]benzoic acid | 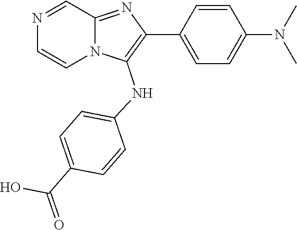 [SMILES: CN(C)c1ccc(-c2nc3cnccn3c2Nc2ccc(C(=O)O)cc2)cc1] |
| P19476 | 34.02 | methyl 3-[(2-phenylimidazo [1,2-a]pyrazin-3- yl)amino]benzoate | 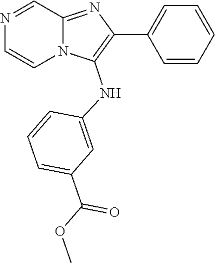 [SMILES: COC(=O)c1cccc(Nc2c(-c3ccccc3)nc3cnccn23)c1] |
| P19477 | 5.91 | 3-[[2-(4-fluorophenyl) imidazo[1,2-a] pyrazin-3-yl]amino] benzoic acid | 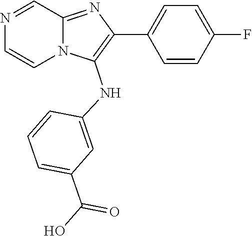 [SMILES: O=C(O)c1cccc(Nc2c(-c3ccc(F)cc3)nc3cnccn23)c1] |
| P19482 | 34.14 | N-methyl-4-[[2-(4- methylphenyl)imidazo [1,2-a]pyrazin-3- yl]amino]benzamide | 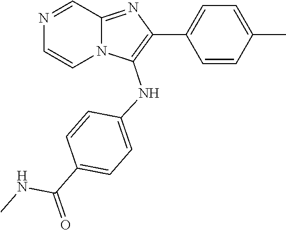 |
| P19483 | 37.84 | 2-(3,4-dimethoxy- phenyl)-N-(2,6-di- methylphenyl)imidazo [1,2-a]pyrazin-3-amine | 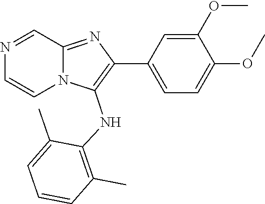 [SMILES: COc1ccc(-c2nc3cnccn3c2Nc2c(C)cccc2C)cc1OC] |
| P19484 | 42.37 | methyl 4-[3-(4-chloroanilino) imidazo[1,2-a] pyrazin-2-yl]benzoate | 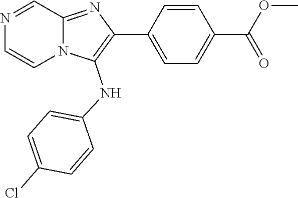 [SMILES: COC(=O)c1ccc(-c2nc3cnccn3c2Nc2ccc(Cl)cc2)cc1] |
| P19485 | 36.03 | methyl 3-[[2-(4-methoxy- phenyl)imidazo[1,2-a] pyrazin-3-yl]amino] benzoate | 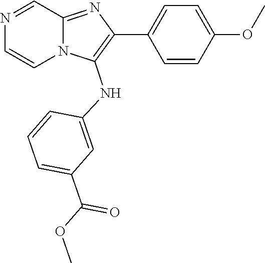 [SMILES: COC(=O)c1cccc(Nc2c(-c3ccc(OC)cc3)nc3cnccn23)c1] |
| P19486 | 40.10 | 3-[[2-(4-methoxy- phenyl)imidazo[1,2-a] pyrazin-3-yl]amino] benzoic acid | 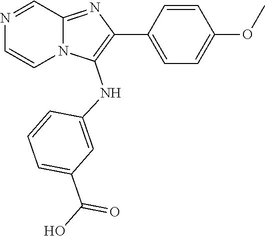 [SMILES: COc1ccc(-c2nc3cnccn3c2Nc2cccc(C(=O)O)c2)cc1] |
| P19487 | 27.30 | 3-[[2-(5-bromo-2- hydroxyphenyl)imidazo [1,2-a]pyrazin-3- yl]amino]benzoic acid | 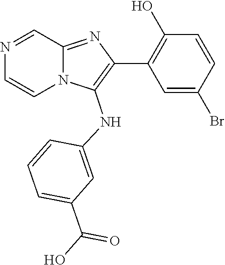 [SMILES: O=C(O)c1cccc(Nc2c(-c3cc(Br)ccc3O)nc3cnccn23)c1] |
| P19492 | 35.36 | 3-[[2-(4-chlorophenyl) imidazo[1,2-a] pyrazin-3-yl]amino] benzamide | 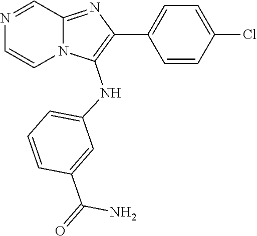 [SMILES: NC(=O)c1cccc(Nc2c(-c3ccc(Cl)cc3)nc3cnccn23)c1] |
| P19493 | 32.29 | 2-(3,4-dimethoxy- phenyl)-N-(4-methoxy- phenyl)imidazo[1,2- a]pyrazin-3-amine | 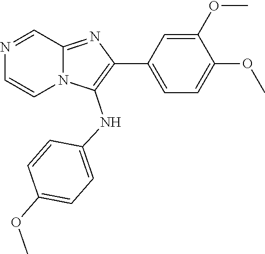 [SMILES: COc1ccc(Nc2c(-c3ccc(OC)c(OC)c3)nc3cnccn23)cc1] |
| P19494 | 25.36 | methyl 3-[[2-(4-methoxy- carbonylphenyl)imidazo [1,2-a]pyrazin-3- yl]amino]benzoate | 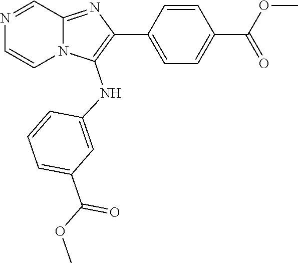 [SMILES: COC(=O)c1ccc(-c2nc3cnccn3c2Nc2cccc(C(=O)OC)c2)cc1] |
| P19495 | 41.69 | methyl 3-[[2-(4-methyl- sulfanylphenyl)imidazo [1,2-a]pyrazin-3- yl]amino]benzoate | 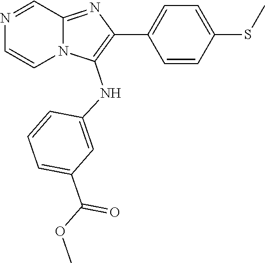 [SMILES: COC(=O)c1cccc(Nc2c(-c3ccc(SC)cc3)nc3cnccn23)c1] |
| P19496 | 40.49 | 3-[[2-(4-hydroxy- phenyl)imidazo[1,2-a] pyrazin-3-yl]amino] benzoic acid | 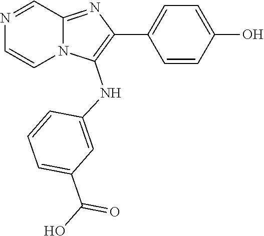 [SMILES: O=C(O)c1cccc(Nc2c(-c3ccc(O)cc3)nc3cnccn23)c1] |
| P19497 | 31.25 | 3-[(2-phenylimidazo [1,2-a]pyrazin-3- yl)amino]benzoic acid | 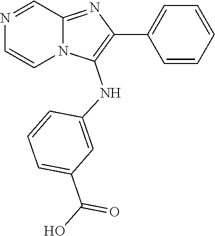 [SMILES: O=C(O)c1cccc(Nc2c(-c3ccccc3)nc3cnccn23)c1] |
| P19502 | 39.08 | 3-[[2-(4-methyl- sulfanylphenyl)imidazo [1,2-a]pyrazin-3- yl]amino]benzamide | 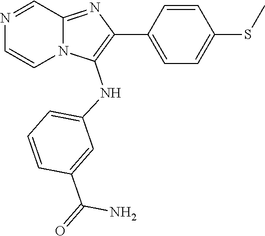 [SMILES: CSc1ccc(-c2nc3cnccn3c2Nc2cccc(C(N)=O)c2)cc1] |
| P19503 | 43.65 | N-(4-chlorophenyl)- 2-(3,4-dimethoxy- phenyl)imidazo[1,2- a]pyrazin-3-amine | 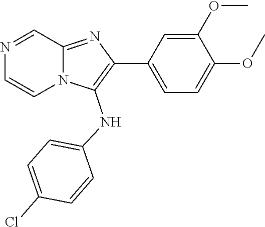 [SMILES: COc1ccc(-c2nc3cnccn3c2Nc2ccc(Cl)cc2)cc1OC] |
| P19504 | 32.56 | ethyl 4-[[2-(4-hydroxy- phenyl)imidazo[1,2-a] pyrazin-3-yl]amino] benzoate | 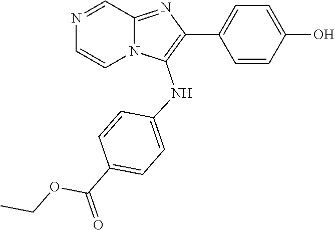 [SMILES: CCOC(=O)c1ccc(Nc2c(-c3ccc(O)cc3)nc3cnccn23)cc1] |
| P19505 | 44.66 | methyl 3-[[2-(4-hydroxy-3- methoxyphenyl)imidazo [1,2-a]pyrazin- 3-yl]amino]benzoate | 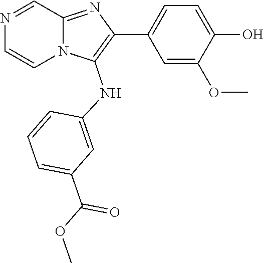 [SMILES: COC(=O)c1cccc(Nc2c(-c3ccc(O)c(OC)c3)nc3cnccn23)c1] |
| P19506 | 34.00 | 3-[[2-(4-hydroxy-3- methoxyphenyl)imidazo [1,2-a]pyrazin- 3-yl]amino]benzoic acid | 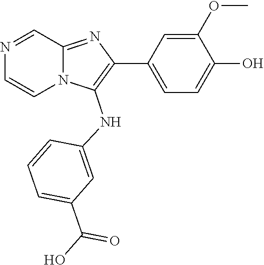 [SMILES: COc1cc(-c2nc3cnccn3c2Nc2cccc(C(=O)O)c2)ccc1O] |
| P19512 | 41.57 | 3-[[2-(4-chlorophenyl) imidazo[1,2-a] pyrazin-3-yl]amino]- N-methylbenzamide | 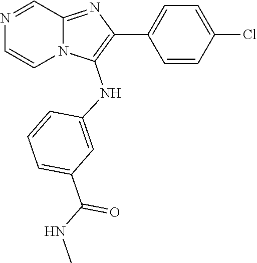 [SMILES: CNC(=O)c1cccc(Nc2c(-c3ccc(Cl)cc3)nc3cnccn23)c1] |
| P19513 | 38.79 | 2-(3,4-dimethoxy- phenyl)-N-(2-ethyl-6- methylphenyl)imidazo [1,2-a]pyrazin- 3-amine | 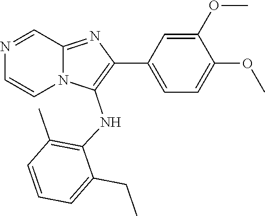 [SMILES: CCc1cccc(C)c1Nc1c(-c2ccc(OC)c(OC)c2)nc2cnccn12] |
| P19514 | 36.02 | 4-[[2-(4-methoxy- phenyl)imidazo[1,2-a] pyrazin-3-yl]amino] benzoic acid | 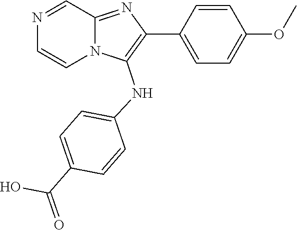 [SMILES: COc1ccc(-c2nc3cnccn3c2Nc2ccc(C(=O)O)cc2)cc1] |
| P19515 | 37.07 | methyl 3-[[2-(4-methyl- phenyl)imidazo[1,2-a] pyrazin-3-yl]amino] benzoate | 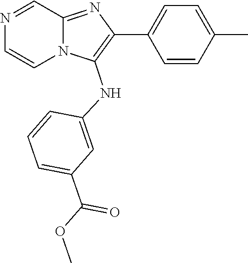 [SMILES: COC(=O)c1cccc(Nc2c(-c3ccc(C)cc3)nc3cnccn23)c1] |
| P19516 | 32.76 | 3-[[2-(4-chlorophenyl) imidazo[1,2-a] pyrazin-3-yl]amino] benzoic acid | 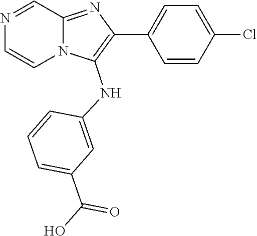 [SMILES: O=C(O)c1cccc(Nc2c(-c3ccc(Cl)cc3)nc3cnccn23)c1] |
| P22402 | 31.33 | 4-[[2-(4-chlorophenyl) imidazo[1,2-a] pyrazin-3-yl]amino]- N-ethylbenzamide | 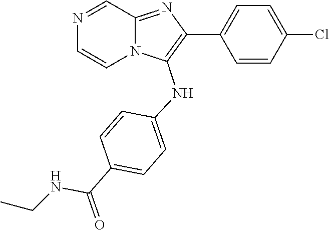 [SMILES: CCNC(=O)c1ccc(Nc2c(-c3ccc(Cl)cc3)nc3cnccn23)cc1] |
| P22403 | 31.69 | 4-[[2-(4-methoxy- phenyl)imidazo[1,2-a] pyrazin-3-yl]amino]- N-(2-morpholin- 4-ylethyl)benzamide | 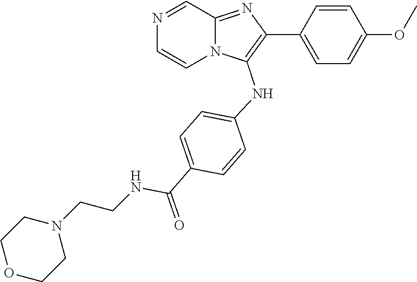 [SMILES: COc1ccc(-c2nc3cnccn3c2Nc2ccc(C(=O)NCCN3CCOCC3)cc2)cc1] |
| P22404 | 26.07 | N-cyclohexyl-4-[(2- phenylimidazo[1,2- a]pyrazin-3-yl)amino] benzamide | 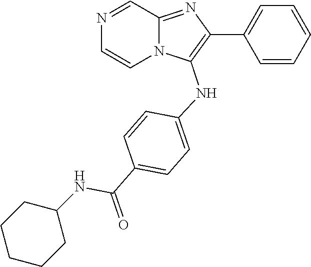 [SMILES: O=C(NC1CCCCC1)c1ccc(Nc2c(-c3ccccc3)nc3cnccn23)cc1] |
| P22405 | 36.46 | [4-[[2-(4-methoxy- phenyl)imidazo[1,2- a]pyrazin-3-yl]amino] phenyl]-morpholin- 4-ylmethanone | 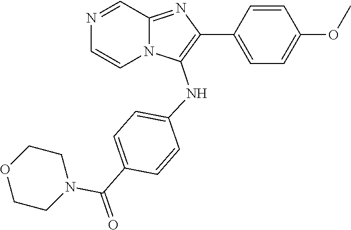 [SMILES: COc1ccc(-c2nc3cnccn3c2Nc2ccc(C(=O)N3CCOCC3)cc2)cc1] |
| P22406 | 37.17 | 3-[[2-[4-(dimethyl- amino)phenyl]imidazo [1,2-a]pyrazin-3- yl]amino]benzamide | 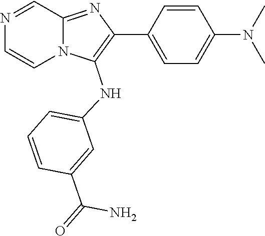 [SMILES: CN(C)c1ccc(-c2nc3cnccn3c2Nc2cccc(C(N)=O)c2)cc1] |
| P22407 | 32.61 | 3-[[2-(4-chlorophenyl) imidazo[1,2-a] pyrazin-3-yl]amino]- N-propan-2- ylbenzamide | 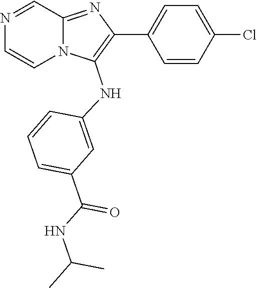 [SMILES: CC(C)NC(=O)c1cccc(Nc2c(-c3ccc(Cl)cc3)nc3cnccn23)c1] |
| P22408 | 17.34 | 3-[[2-(4-chlorophenyl) imidazo[1,2-a] pyrazin-3-yl]amino]- N-(oxolan-2- ylmethyl)benzamide | 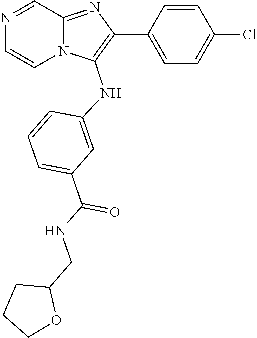 [SMILES: O=C(NCC1CCCO1)c1cccc(Nc2c(-c3ccc(Cl)cc3)nc3cnccn23)c1] |
| P22409 | 28.25 | morpholin-4-yl-[3- [(2-phenylimidazo [1,2-a]pyrazin-3-yl) amino]phenyl] methanone | 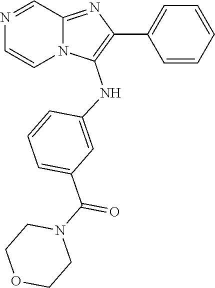 [SMILES: O=C(c1cccc(Nc2c(-c3ccccc3)nc3cnccn23)c1)N1CCOCC1] |
| P22410 | 42.19 | 4-[[2-(3,4-dimethoxy- phenyl)imidazo[1,2- a]pyrazin-3-yl] amino]-N- ethylbenzamide | 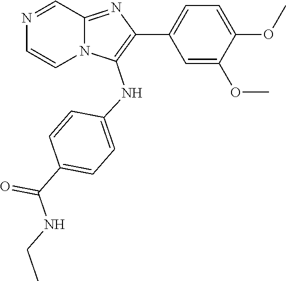 [SMILES: CCNC(=O)c1ccc(Nc2c(-c3ccc(OC)c(OC)c3)nc3cnccn23)cc1] |
| P22412 | 33.45 | N-ethyl-4-[[2-(4- methylphenyl)imidazo [1,2-a]pyrazin-3- yl]amino]benzamide | 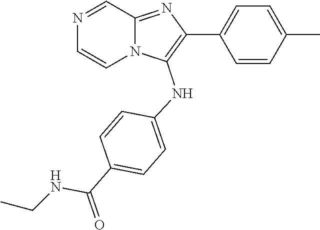 [SMILES: CCNC(=O)c1ccc(Nc2c(-c3ccc(C)cc3)nc3cnccn23)cc1] |
| P22413 | 36.07 | 4-[[2-(4-chlorophenyl) imidazo[1,2-a] pyrazin-3-yl]amino]- N-(2-morpholin-4- ylethyl)benzamide | 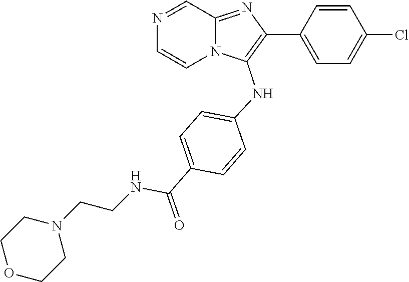 [SMILES: O=C(NCCN1CCOCC1)c1ccc(Nc2c(-c3ccc(Cl)cc3)nc3cnccn23)cc1] |
| P22414 | 51.11 | N-cyclohexyl-4-[[2- (4-methoxyphenyl) imidazo[1,2-a]pyrazin- 3-yl]amino]benzamide | 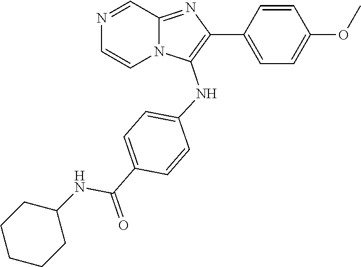 [SMILES: COc1ccc(-c2nc3cnccn3c2Nc2ccc(C(=O)NC3CCCCC3)cc2)cc1] |
| P22415 | 63.34 | [4-[[2-(4-methyl- phenyl)imidazo[1,2-a] pyrazin-3-yl]amino] phenyl]-morpholin- 4-ylmethanone | 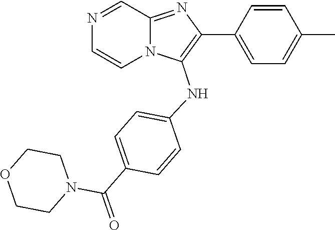 [SMILES: Cc1ccc(-c2nc3cnccn3c2Nc2ccc(C(=O)N3CCOCC3)cc2)cc1] |
| P22416 | 30.18 | N-methyl-3-[(2- phenylimidazo[1,2-a] pyrazin-3-yl)amino] benzamide | 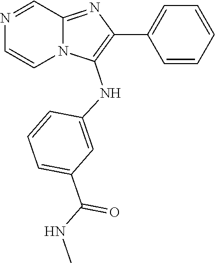 [SMILES: CNC(=O)c1cccc(Nc2c(-c3ccccc3)nc3cnccn23)c1] |
| P22417 | 43.38 | 3-[[2-(4-methyl- sulfanylphenyl)imidazo [1,2-a]pyrazin-3- yl]amino]-N-propan- 2-ylbenzamide | 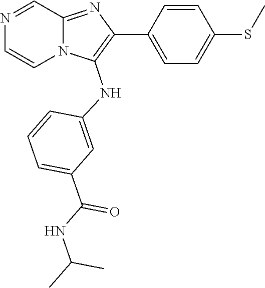 [SMILES: CSc1ccc(-c2nc3cnccn3c2Nc2cccc(C(=O)NC(C)C)c2)cc1] |
| P22418 | 34.55 | 3-[[2-(4-methyl- sulfanylphenyl)imidazo [1,2-a]pyrazin-3- yl]amino]-N-(oxolan- 2-ylmethyl)benzamide | 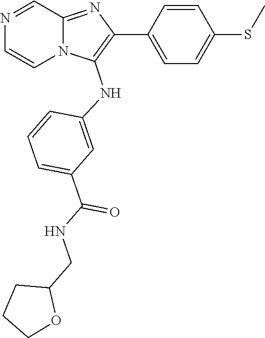 [SMILES: CSc1ccc(-c2nc3cnccn3c2Nc2cccc(C(=O)NCC3CCCO3)c2)cc1] |
| P22420 | 43.91 | 4-[[2-(3,4-dimethoxy- phenyl)imidazo[1,2- a]pyrazin-3-yl] amino]-N-propan-2- ylbenzamide | 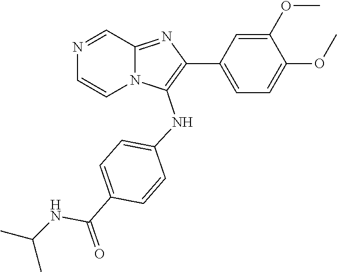 [SMILES: COc1ccc(-c2nc3cnccn3c2Nc2ccc(C(=O)NC(C)C)cc2)cc1OC] |
| P22425 | 40.71 | [4-[[2-(4-methyl- sulfanylphenyl)imidazo [1,2-a]pyrazin-3- yl]amino]phenyl]- morpholin-4- ylmethanone | 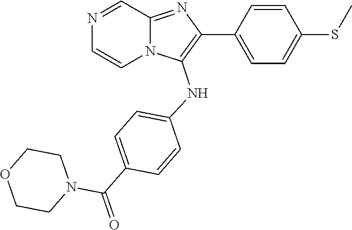 [SMILES: CSc1ccc(-c2nc3cnccn3c2Nc2ccc(C(=O)N3CCOCC3)cc2)cc1] |
| P22427 | 22.79 | N-[2-(1,4-oxazinan- 4-yl)ethyl]-3-[(2- phenylimidazo[1,2- a]pyrazin-3-yl)amino] benzamide | 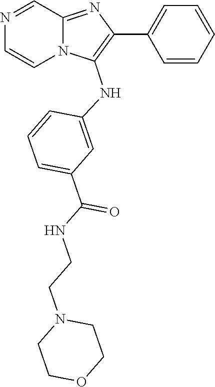 [SMILES: O=C(NCCN1CCOCC1)c1cccc(Nc2c(-c3ccccc3)nc3cnccn23)c1] |
| P22429 | 24.72 | [3-[[2-(4-chlorophenyl) imidazo[1,2-a] pyrazin-3-yl]amino] phenyl]-morpholin- 4-ylmethanone | 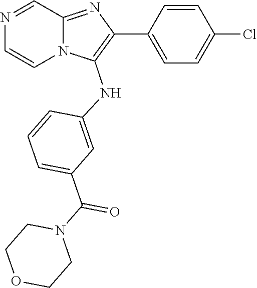 [SMILES: O=C(c1cccc(Nc2c(-c3ccc(Cl)cc3)nc3cnccn23)c1)N1CCOCC1] |
| P22432 | 42.24 | 4-[[2-(4-methoxy- phenyl)imidazo[1,2-a] pyrazin-3-yl]amino]- N-propan-2- ylbenzamide | 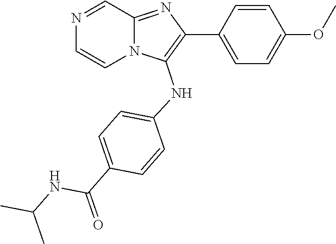 [SMILES: COc1ccc(-c2nc3cnccn3c2Nc2ccc(C(=O)NC(C)C)cc2)cc1] |
| P22433 | 30.54 | N-[2-(dimethylamino) ethyl]-4-[(2-phenyl- imidazo[1,2-a] pyrazin-3-yl)amino] benzamide | 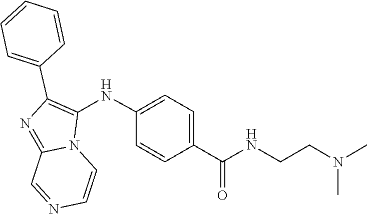 [SMILES: CN(C)CCNC(=O)c1ccc(Nc2c(-c3ccccc3)nc3cnccn23)cc1] |
| P22434 | 41.13 | N-cyclohexyl-4-[[2- (4-methylphenyl) imidazo[1,2-a]pyrazin- 3-yl]amino]benzamide | 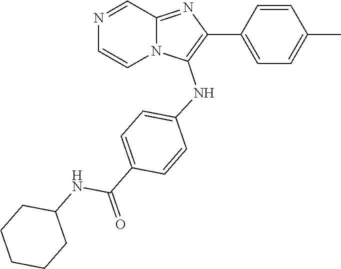 [SMILES: Cc1ccc(-c2nc3cnccn3c2Nc2ccc(C(=O)NC3CCCCC3)cc2)cc1] |
| P22435 | 23.95 | N-(furan-2-ylmethyl)- 4-[(2-phenylimidazo [1,2-a]pyrazin- 3-yl)amino]benzamide | 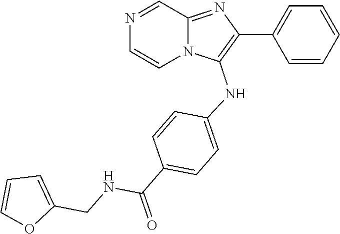 [SMILES: O=C(NCc1ccco1)c1ccc(Nc2c(-c3ccccc3)nc3cnccn23)cc1] |
| P22436 | 31.30 | N-methyl-3-[[2-(4- methylsulfanylphenyl) imidazo[1,2-a] pyrazin-3-yl]amino] benzamide | 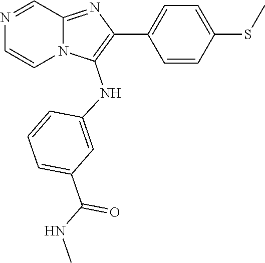 [SMILES: CNC(=O)c1cccc(Nc2c(-c3ccc(SC)cc3)nc3cnccn23)c1] |
| P22437 | 24.22 | 3-[[2-(4-methoxy- phenyl)imidazo[1,2- a]pyrazin-3-yl]amino]- N-(2-morpholin- 4-ylethyl)benzamide | 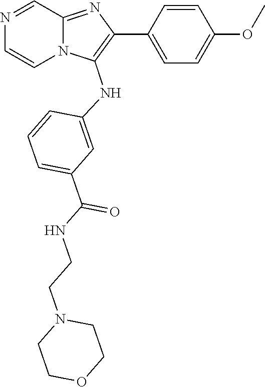 [SMILES: COc1ccc(-c2nc3cnccn3c2Nc2cccc(C(=O)NCCN3CCOCC3)c2)cc1] |
| P22438 | 54.25 | 3-[[2-(4-chlorophenyl) imidazo[1,2-a] pyrazin-3-yl]amino]- N-cyclohexyl- benzamide | 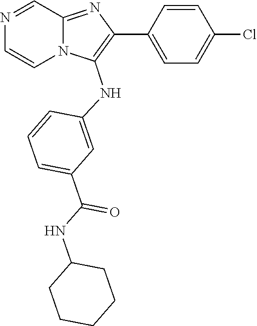 [SMILES: O=C(NC1CCCCC1)c1cccc(Nc2c(-c3ccc(Cl)cc3)nc3cnccn23)c1] |
| P22439 | 23.21 | [3-[[2-(4-methyl- sulfanylphenyl)imidazo [1,2-a]pyrazin-3- yl]amino]phenyl]- morpholin-4- ylmethanone | 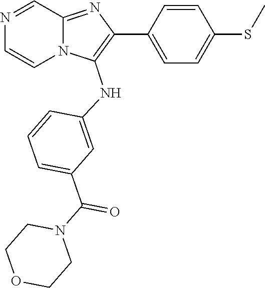 [SMILES: CSc1ccc(-c2nc3cnccn3c2Nc2cccc(C(=O)N3CCOCC3)c2)cc1] |
| P22440 | 33.48 | 4-[[2-(3,4-dimethoxy- phenyl)imidazo[1,2- a]pyrazin-3-yl] amino]-N-(oxolan-2- ylmethyl)benzamide | 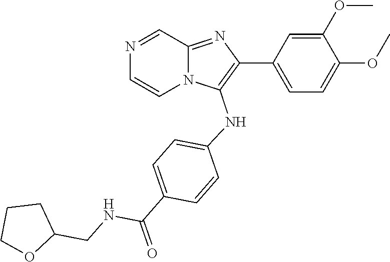 [SMILES: COc1ccc(-c2nc3cnccn3c2Nc2ccc(C(=O)NCC3CCCO3)cc2)cc1OC] |
| P22442 | 37.11 | 4-[[2-(4-chlorophenyl) imidazo[1,2-a] pyrazin-3-yl]amino]- N-propan-2- ylbenzamide | 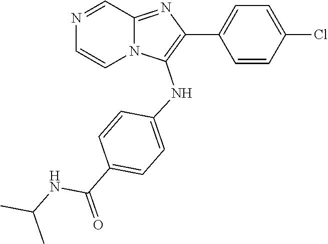 [SMILES: CC(C)NC(=O)c1ccc(Nc2c(-c3ccc(Cl)cc3)nc3cnccn23)cc1] |
| P22443 | 41.16 | N-[2-(dimethylamino) ethyl]-4-[[2-(4- methoxyphenyl) imidazo[1,2-a]pyrazin- 3-yl]amino]benzamide | 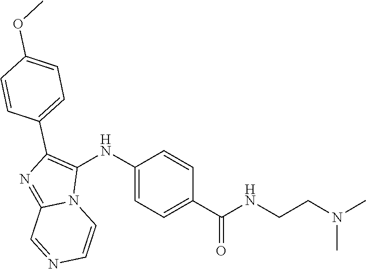 [SMILES: COc1ccc(-c2nc3cnccn3c2Nc2ccc(C(=O)NCCN(C)C)cc2)cc1] |
| P22444 | 43.91 | [4-[(2-phenylimidazo [1,2-a]pyrazin-3- yl)amino]phenyl]- piperidin-1- ylmethanone | 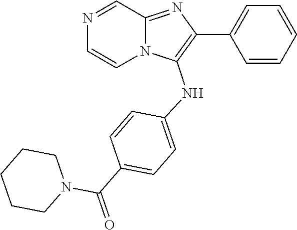 [SMILES: O=C(c1ccc(Nc2c(-c3ccccc3)nc3cnccn23)cc1)N1CCCCC1] |
| P22445 | 62.30 | N-(furan-2-ylmethyl)- 4-[[2-(4-methoxy- phenyl)imidazo[1,2- a]pyrazin-3-yl] amino]benzamide | 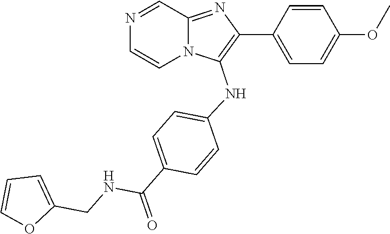 [SMILES: COc1ccc(-c2nc3cnccn3c2Nc2ccc(C(=O)NCc3ccco3)cc2)cc1] |
| P22446 | 44.36 | N-ethyl-3-[(2-phenyl- imidazo[1,2-a] pyrazin-3-yl)amino] benzamide | 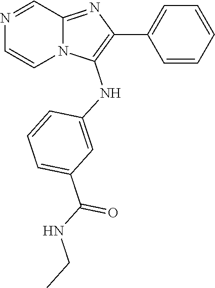 [SMILES: CCNC(=O)c1cccc(Nc2c(-c3ccccc3)nc3cnccn23)c1] |
| P22447 | 41.92 | 3-[[2-(4-chlorophenyl) imidazo[1,2-a] pyrazin-3-yl]amino]- N-(2-morpholin-4- ylethyl)benzamide | 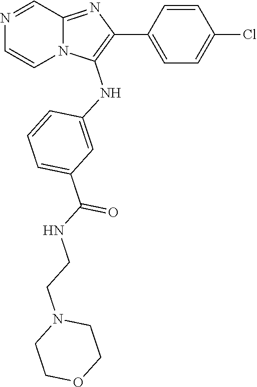 [SMILES: O=C(NCCN1CCOCC1)c1cccc(Nc2c(-c3ccc(Cl)cc3)nc3cnccn23)c1] |
| P22448 | 26.86 | N-cyclohexyl-3-[[2- (4-methylsulfanyl- phenyl)imidazo[1,2- a]pyrazin-3-yl]amino] benzamide | 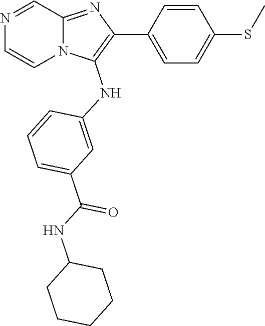 [SMILES: CSc1ccc(-c2nc3cnccn3c2Nc2cccc(C(=O)NC3CCCCC3)c2)cc1] |
| P22449 | 27.46 | N-(furan-2-ylmethyl)- 3-[(2-phenylimidazo [1,2-a]pyrazin- 3-yl)amino]benzamide | 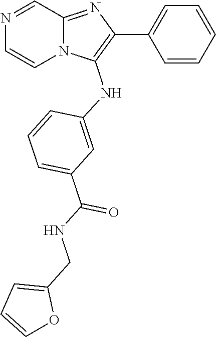 [SMILES: O=C(NCc1ccco1)c1cccc(Nc2c(-c3ccccc3)nc3cnccn23)c1] |
| P22450 | 20.76 | N-cyclohexyl-4-[[2- (3,4-dimethoxyphenyl) imidazo[1,2-a] pyrazin-3-yl]amino] benzamide | 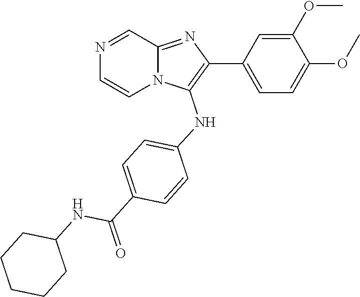 [SMILES: COc1ccc(-c2nc3cnccn3c2Nc2ccc(C(=O)NC3CCCCC3)cc2)cc1OC] |
| P22451 | 34.67 | N-methyl-4-[(2-phenyl- imidazo[1,2-a] pyrazin-3-yl)amino] benzamide | 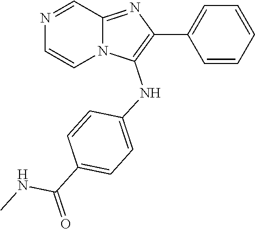 [SMILES: CNC(=O)c1ccc(Nc2c(-c3ccccc3)nc3cnccn23)cc1] |
| P22452 | 33.65 | 4-[[2-(4-methylphenyl) imidazo[1,2-a] pyrazin-3-yl]amino]- N-propan-2- ylbenzamide | 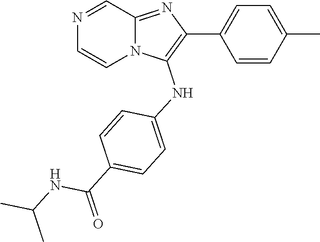 [SMILES: Cc1ccc(-c2nc3cnccn3c2Nc2ccc(C(=O)NC(C)C)cc2)cc1] |
| P22453 | 36.58 | N-[2-(dimethylamino) ethyl]-4-[[2-(4- methylphenyl)imidazo [1,2-a]pyrazin-3- yl]amino]benzamide | 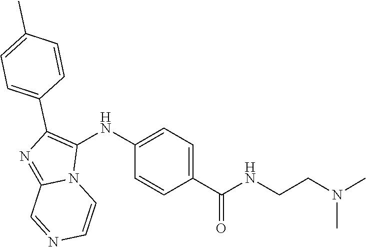 [SMILES: Cc1ccc(-c2nc3cnccn3c2Nc2ccc(C(=O)NCCN(C)C)cc2)cc1] |
| P22454 | 31.75 | [4-[[2-(4-methoxy- phenyl)imidazo[1,2- a]pyrazin-3-yl]amino] phenyl]-piperidin- 1-ylmethanone | 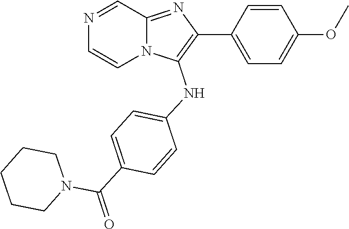 [SMILES: COc1ccc(-c2nc3cnccn3c2Nc2ccc(C(=O)N3CCCCC3)cc2)cc1] |
| P22455 | 40.15 | N-(furan-2-ylmethyl)- 4-[[2-(4-methyl- phenyl)imidazo[1,2- a]pyrazin-3-yl]amino] benzamide | 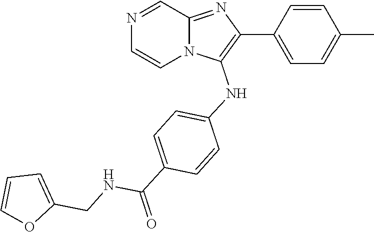 [SMILES: Cc1ccc(-c2nc3cnccn3c2Nc2ccc(C(=O)NCc3ccco3)cc2)cc1] |
| P22456 | 30.28 | 3-[[2-(4-chlorophenyl) imidazo[1,2-a] pyrazin-3-yl]amino]- N-ethylbenzamide | 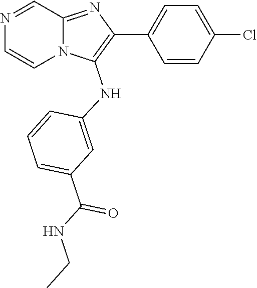 [SMILES: CCNC(=O)c1cccc(Nc2c(-c3ccc(Cl)cc3)nc3cnccn23)c1] |
| P22457 | 38.63 | 3-[[2-(4-chlorophenyl) imidazo[1,2-a] pyrazin-3-yl]amino]- N-[2-(dimethylamino) ethyl]benzamide | 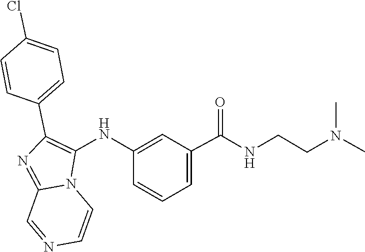 [SMILES: CN(C)CCNC(=O)c1cccc(Nc2c(-c3ccc(Cl)cc3)nc3cnccn23)c1] |
| P22458 | 35.45 | [3-[(2-phenylimidazo [1,2-a]pyrazin-3- yl)amino]phenyl]- piperidin-1- ylmethanone | 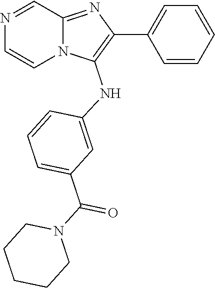 [SMILES: O=C(c1cccc(Nc2c(-c3ccccc3)nc3cnccn23)c1)N1CCCCC1] |
| P22459 | 35.42 | N-(furan-2-ylmethyl)- 3-[[2-(4-methoxy- phenyl)imidazo[1,2- a]pyrazin-3-yl] amino]benzamide | 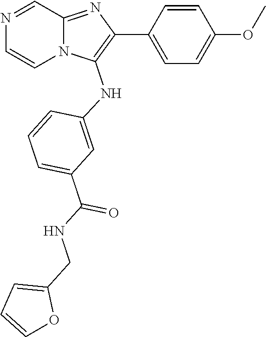 [SMILES: COc1ccc(-c2nc3cnccn3c2Nc2cccc(C(=O)NCc3ccco3)c2)cc1] |
| P22460 | 31.76 | [4-[[2-(3,4-dimethoxy- phenyl)imidazo[1,2- a]pyrazin-3-yl] amino]phenyl]- piperidin-1- ylmethanone | 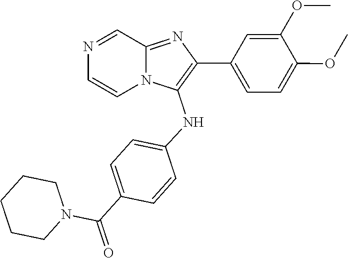 [SMILES: COc1ccc(-c2nc3cnccn3c2Nc2ccc(C(=O)N3CCCCC3)cc2)cc1OC] |
| P22461 | 32.32 | N-ethyl-4-[(2-phenyl- imidazo[1,2-a] pyrazin-3-yl)amino] benzamide | 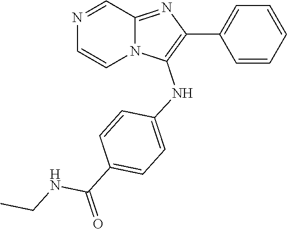 [SMILES: CCNC(=O)c1ccc(Nc2c(-c3ccccc3)nc3cnccn23)cc1] |
| P22462 | 40.68 | 4-[[2-(4-methyl- sulfanylphenyl)imidazo [1,2-a]pyrazin-3- yl]amino]-N-propan- 2-ylbenzamide | 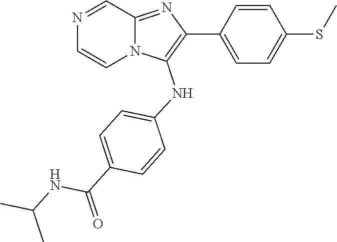 [SMILES: CSc1ccc(-c2nc3cnccn3c2Nc2ccc(C(=O)NC(C)C)cc2)cc1] |
| P22463 | 25.80 | N-(oxolan-2-ylmethyl)- 4-[(2-phenylimidazo [1,2-a]pyrazin-3- yl)amino]benzamide | 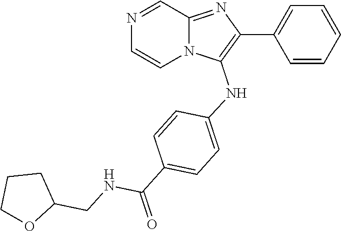 [SMILES: O=C(NCC1CCCO1)c1ccc(Nc2c(-c3ccccc3)nc3cnccn23)cc1] |
| P22464 | 29.64 | [4-[[2-(4-methyl- phenyl)imidazo[1,2- a]pyrazin-3-yl]amino] phenyl]-piperidin- 1-ylmethanone | 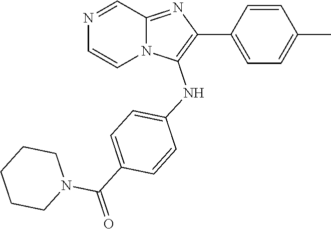 [SMILES: Cc1ccc(-c2nc3cnccn3c2Nc2ccc(C(=O)N3CCCCC3)cc2)cc1] |
| P22465 | 29.63 | N-(furan-2-ylmethyl)- 4-[[2-(4-methyl- sulfanylphenyl)imidazo [1,2-a]pyrazin- 3-yl]amino]benzamide | 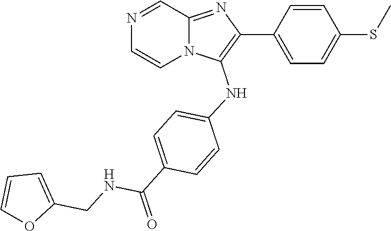 [SMILES: CSc1ccc(-c2nc3cnccn3c2Nc2ccc(C(=O)NCc3ccco3)cc2)cc1] |
| P22466 | 22.16 | N-ethyl-3-[[2-(4- methylsulfanylphenyl) imidazo[1,2-a] pyrazin-3-yl]amino] benzamide | 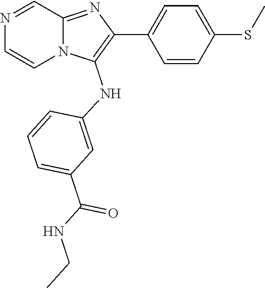 [SMILES: CCNC(=O)c1cccc(Nc2c(-c3ccc(SC)cc3)nc3cnccn23)c1] |
| P22467 | 29.82 | N-[2-(dimethylamino) ethyl]-3-[[2-(4- methylsulfanylphenyl) imidazo[1,2-a] pyrazin-3-yl]amino] benzamide | 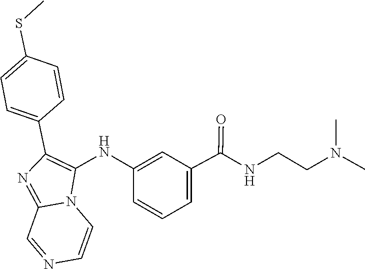 [SMILES: CSc1ccc(-c2nc3cnccn3c2Nc2cccc(C(=O)NCCN(C)C)c2)cc1] |
| P22468 | 29.03 | [3-[[2-(4-chlorophenyl) imidazo[1,2-a] pyrazin-3-yl]amino] phenyl]-piperidin- 1-ylmethanone | 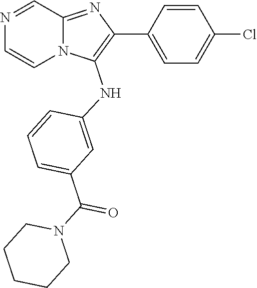 [SMILES: O=C(c1cccc(Nc2c(-c3ccc(Cl)cc3)nc3cnccn23)c1)N1CCCCC1] |
| P22469 | 40.52 | 3-[[2-(4-chlorophenyl) imidazo[1,2-a] pyrazin-3-yl]amino]- N-(furan-2-ylmethyl) benzamide | 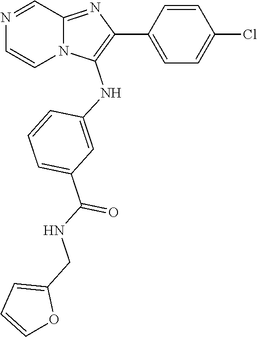 [SMILES: O=C(NCc1ccco1)c1cccc(Nc2c(-c3ccc(Cl)cc3)nc3cnccn23)c1] |
| P22470 | 32.13 | [4-[[2-(3,4-dimethoxy- phenyl)imidazo[1,2- a]pyrazin-3-yl] amino]phenyl]- morpholin-4- ylmethanone | 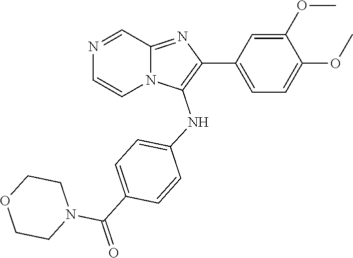 [SMILES: COc1ccc(-c2nc3cnccn3c2Nc2ccc(C(=O)N3CCOCC3)cc2)cc1OC] |
| P22472 | 32.98 | N-(2-morpholin-4- ylethyl)-4-[(2-phenyl- imidazo[1,2-a] pyrazin-3-yl)amino] benzamide | 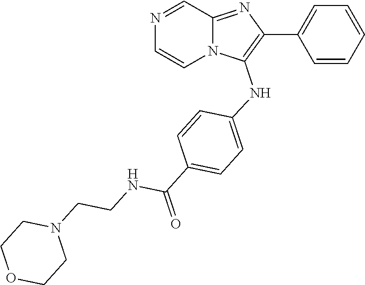 [SMILES: O=C(NCCN1CCOCC1)c1ccc(Nc2c(-c3ccccc3)nc3cnccn23)cc1] |
| P22473 | 36.81 | 4-[[2-(4-methoxy- phenyl)imidazo[1,2-a] pyrazin-3-yl]amino]- N-(oxolan-2-ylmethyl) benzamide | 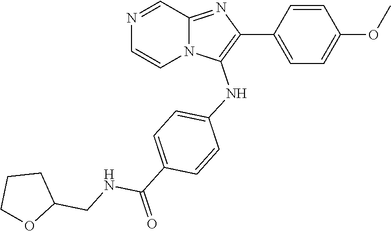 [SMILES: COc1ccc(-c2nc3cnccn3c2Nc2ccc(C(=O)NCC3CCCO3)cc2)cc1] |
| P22474 | 35.32 | morpholin-4-yl-[4- [(2-phenylimidazo [1,2-a]pyrazin-3-yl) amino]phenyl] methanone | 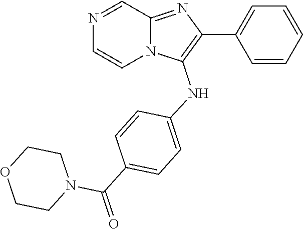 [SMILES: O=C(c1ccc(Nc2c(-c3ccccc3)nc3cnccn23)cc1)N1CCOCC1] |
| P22475 | 60.09 | 3-[[2-(4-methoxy- phenyl)imidazo[1,2- a]pyrazin-3-yl]amino] benzamide | 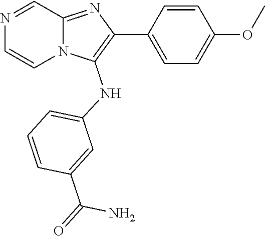 [SMILES: COc1ccc(-c2nc3cnccn3c2Nc2cccc(C(N)=O)c2)cc1] |
| P22476 | 28.98 | 3-[(2-phenylimidazo [1,2-a]pyrazin-3- yl)amino]-N-propan- 2-ylbenzamide | 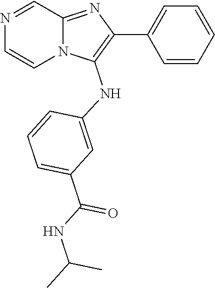 [SMILES: CC(C)NC(=O)c1cccc(Nc2c(-c3ccccc3)nc3cnccn23)c1] |
| P22477 | 40.96 | N-(oxolan-2-ylmethyl)- 3-[(2-phenylimidazo [1,2-a]pyrazin-3- yl)amino]benzamide | 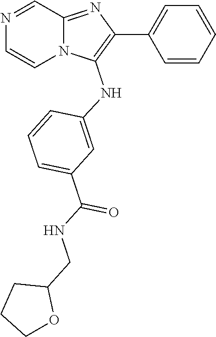 [SMILES: O=C(NCC1CCCO1)c1cccc(Nc2c(-c3ccccc3)nc3cnccn23)c1] |
| P22478 | 43.92 | [3-[[2-(4-methyl- sulfanylphenyl)imidazo [1,2-a]pyrazin-3- yl]amino]phenyl]- piperidin-1- ylmethanone | 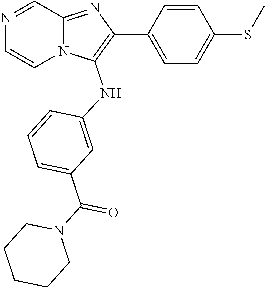 [SMILES: CSc1ccc(-c2nc3cnccn3c2Nc2cccc(C(=O)N3CCCCC3)c2)cc1] |
| P22480 | 40.62 | 4-[[2-(3,4-dimethoxy- phenyl)imidazo[1,2- a]pyrazin-3-yl] amino]-N-(furan-2- ylmethyl)benzamide | 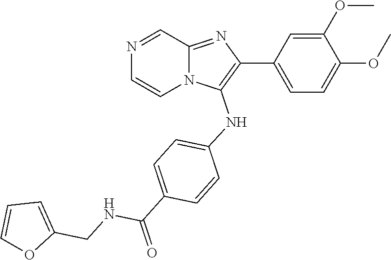 [SMILES: COc1ccc(-c2nc3cnccn3c2Nc2ccc(C(=O)NCc3ccco3)cc2)cc1OC] |
| P22481 | 11.79 | N-cyclohexyl-3-[[2- (4-fluorophenyl) imidazo[1,2-a] pyrazin-3-yl]amino] benzamide | 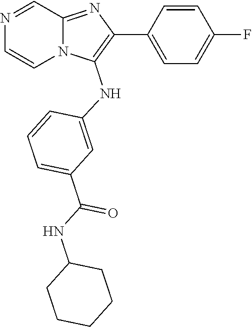 [SMILES: O=C(NC1CCCCC1)c1cccc(Nc2c(-c3ccc(F)cc3)nc3cnccn23)c1] |
| P22482 | 22.17 | ethyl 4-[[2-(4-methylphenyl) imidazo[1,2-a] pyrazin-3-yl]amino] benzoate | 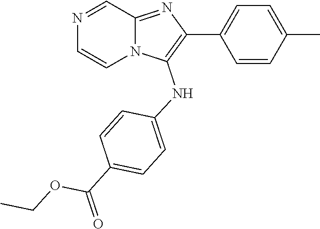 [SMILES: CCOC(=O)c1ccc(Nc2c(-c3ccc(C)cc3)nc3cnccn23)cc1] |
| P22483 | 29.42 | ethyl 4-[(2-phenylimidazo [1,2-a]pyrazin-3- yl)amino]benzoate | 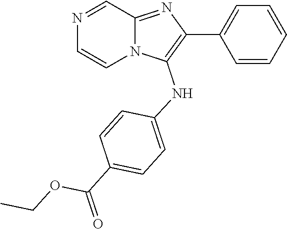 [SMILES: CCOC(=O)c1ccc(Nc2c(-c3ccccc3)nc3cnccn23)cc1] |
| P22491 | 22.37 | methyl 4-[3-(3,4-dimethoxy- anilino)imidazo[1,2- a]pyrazin-2-yl] benzoate | 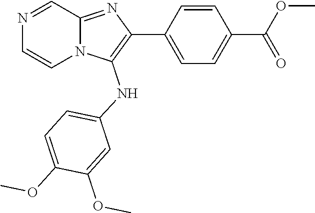 |
| P22492 | 29.49 | ethyl 4-[[2-(4-chlorophenyl) imidazo[1,2-a] pyrazin-3-yl]amino] benzoate | 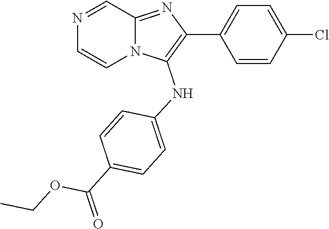 [SMILES: CCOC(=O)c1ccc(Nc2c(-c3ccc(Cl)cc3)nc3cnccn23)cc1] |
| P22493 | 13.18 | 4-[[2-(3-chlorophenyl) imidazo[1,2-a] pyrazin-3-yl]amino] benzoic acid | 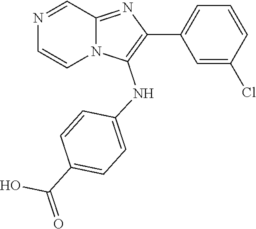 [SMILES: O=C(O)c1ccc(Nc2c(-c3cccc(Cl)c3)nc3cnccn23)cc1] |
| P22501 | 28.74 | 4-[3-(2-ethyl-6-methyl- anilino)imidazo [1,2-a]pyrazin-2- yl]-N-methyl- benzamide | 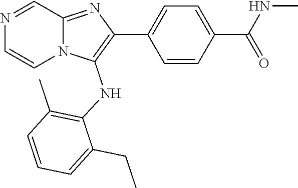 [SMILES: CCc1cccc(C)c1Nc1c(-c2ccc(C(=O)NC)cc2)nc2cnccn12] |
| P22502 | 14.38 | ethyl 4-[[2-[4-(dimethyl- amino)phenyl]imidazo [1,2-a]pyrazin-3- yl]amino]benzoate | 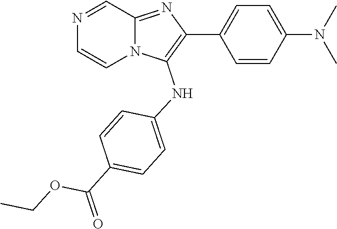 [SMILES: CCOC(=O)c1ccc(Nc2c(-c3ccc(N(C)C)cc3)nc3cnccn23)cc1] |
| P22503 | 27.58 | 4-[[2-(5-bromo-2- hydroxyphenyl)imidazo [1,2-a]pyrazin-3- yl]amino]benzoic acid | 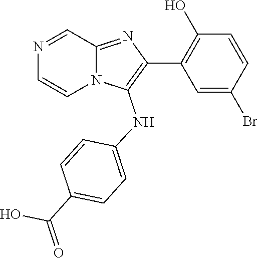 [SMILES: O=C(O)c1ccc(Nc2c(-c3cc(Br)ccc3O)nc3cnccn23)cc1] |
| P22511 | 27.73 | N-ethyl-4-[3-(2- ethyl-6-methylanilino) imidazo[1,2-a] pyrazin-2-yl]benzamide | 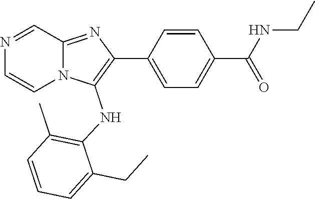 [SMILES: CCNC(=O)c1ccc(-c2nc3cnccn3c2Nc2c(C)cccc2CC)cc1] |
| P22512 | 26.94 | ethyl 4-[[2-(2-hydroxy- phenyl)imidazo[1,2- a]pyrazin-3-yl]amino] benzoate | 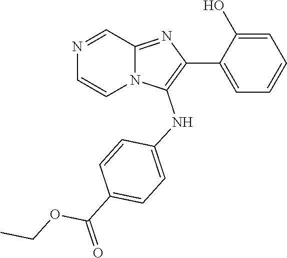 [SMILES: CCOC(=O)c1ccc(Nc2c(-c3ccccc3O)nc3cnccn23)cc1] |
| P22513 | 21.93 | N-[4-[3-(2-ethyl-6- methylanilino)imidazo [1,2-a]pyrazin- 2-yl]phenyl]acetamide | 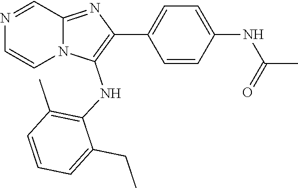 [SMILES: CCc1cccc(C)c1Nc1c(-c2ccc(NC(C)=O)cc2)nc2cnccn12] |
| P22521 | 21.97 | methyl 4-[3-(4-ethoxy- carbonylanilino) imidazo[1,2-a]pyrazin- 2-yl]benzoate | 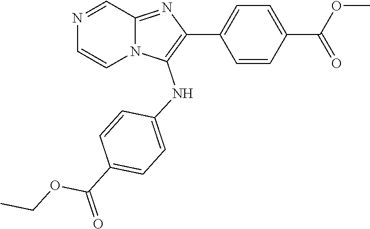 [SMILES: CCOC(=O)c1ccc(Nc2c(-c3ccc(C(=O)OC)cc3)nc3cnccn23)cc1] |
| P22522 | 32.27 | ethyl 4-[[2-(3-chlorophenyl) imidazo[1,2-a] pyrazin-3-yl]amino] benzoate | 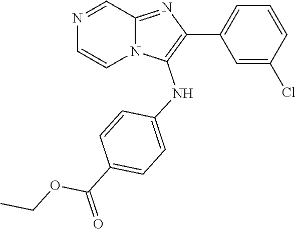 [SMILES: CCOC(=O)c1ccc(Nc2c(-c3cccc(Cl)c3)nc3cnccn23)cc1] |
| P22531 | 31.91 | ethyl 4-[[2-(4-methoxy- phenyl)imidazo[1,2- a]pyrazin-3-yl]amino] benzoate | 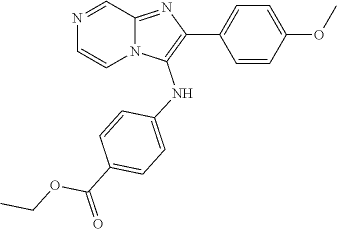 [SMILES: CCOC(=O)c1ccc(Nc2c(-c3ccc(OC)cc3)nc3cnccn23)cc1] |
| P22532 | 24.18 | ethyl 4-[[2-(3-bromophenyl) imidazo[1,2-a] pyrazin-3-yl]amino] benzoate | 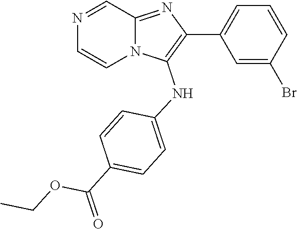 [SMILES: CCOC(=O)c1ccc(Nc2c(-c3cccc(Br)c3)nc3cnccn23)cc1] |
| P22541 | 33.76 | ethyl 4-[[2-(4-methyl- sulfanylphenyl)imidazo [1,2-a]pyrazin-3- yl]amino]benzoate | 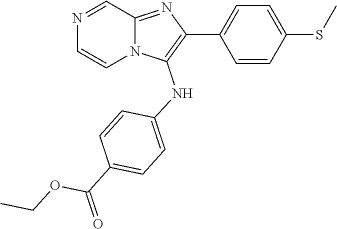 [SMILES: CCOC(=O)c1ccc(Nc2c(-c3ccc(SC)cc3)nc3cnccn23)cc1] |
| P22542 | 31.48 | ethyl 4-[[2-(5-bromo-2- hydroxyphenyl)imidazo [1,2-a]pyrazin-3- yl]amino]benzoate | 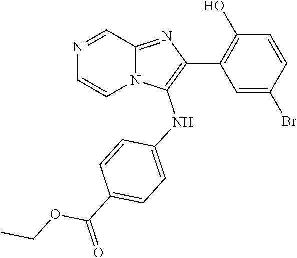 [SMILES: CCOC(=O)c1ccc(Nc2c(-c3cc(Br)ccc3O)nc3cnccn23)cc1] |
| P22551 | 20.96 | ethyl 4-[[2-(4-hydroxy-3- methoxyphenyl) imidazo[1,2-a]pyrazin- 3-yl]amino]benzoate | 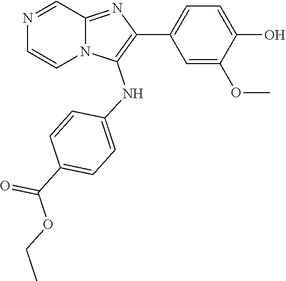 [SMILES: CCOC(=O)c1ccc(Nc2c(-c3ccc(O)c(OC)c3)nc3cnccn23)cc1] |
| P22552 | 19.90 | ethyl 4-[[2-(3-hydroxy- phenyl)imidazo[1,2- a]pyrazin-3-yl]amino] benzoate | 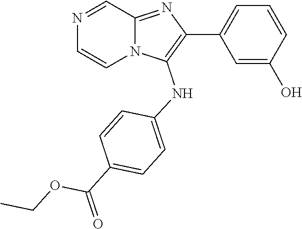 [SMILES: CCOC(=O)c1ccc(Nc2c(-c3cccc(O)c3)nc3cnccn23)cc1] |
| P24359 | 37.10 | methyl 4-[3-(cyclohexylamino) imidazo[1,2-a] pyrazin-2-yl]benzoate | 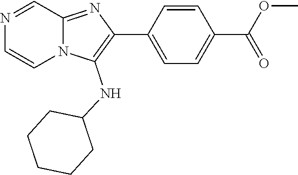 [SMILES: COC(=O)c1ccc(-c2nc3cnccn3c2NC2CCCCC2)cc1] |
| P24803 | 24.71 | ethyl 4-[[2-(4-morpholin- 4-ylphenyl)imidazo [1,2-a]pyrazin-3- yl]amino]benzoate | 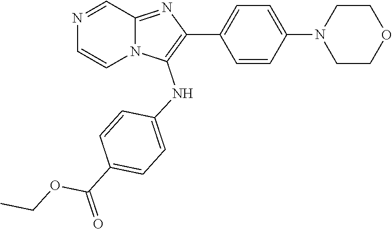 [SMILES: CCOC(=O)c1ccc(Nc2c(-c3ccc(N4CCOCC4)cc3)nc3cnccn23)cc1] |
| P24813 | 31.93 | 4-[[2-(4-morpholin- 4-ylphenyl)imidazo [1,2-a]pyrazin-3- yl]amino]benzoic acid | 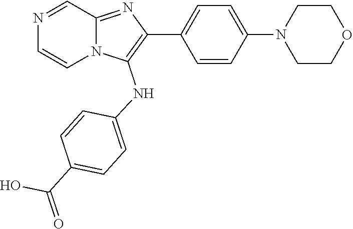 [SMILES: O=C(O)c1ccc(Nc2c(-c3ccc(N4CCOCC4)cc3)nc3cnccn23)cc1] |
| P24823 | 31.23 | methyl 3-[[2-(4-nitrophenyl) imidazo[1,2-a] pyrazin-3-yl]amino] benzoate | 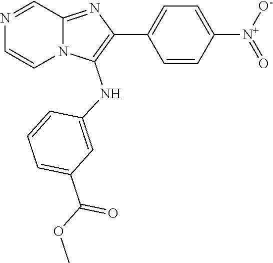 [SMILES: COC(=O)c1cccc(Nc2c(-c3ccc([N+](=O)[O-])cc3)nc3cnccn23)c1] |
| P24833 | 35.11 | 3-[[2-(4-morpholin- 4-ylphenyl)imidazo [1,2-a]pyrazin-3- yl]amino]benzoic acid | 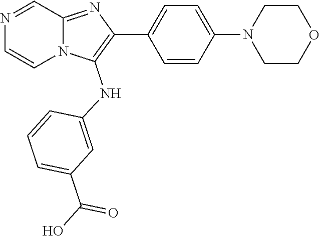 [SMILES: O=C(O)c1cccc(Nc2c(-c3ccc(N4CCOCC4)cc3)nc3cnccn23)c1] |
| P24852 | 24.86 | N-(2,3-dihydro-1,4- benzodioxin-6-yl)- 2-(3,4-dimethoxy- phenyl)imidazo[1,2- a]pyrazin-3-amine | 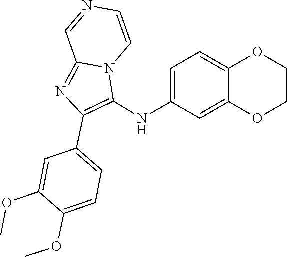 [SMILES: COc1ccc(-c2nc3cnccn3c2Nc2ccc3c(c2)OCCO3)cc1OC] |
| P24862 | 30.12 | methyl 4-[3-(2,3-dihydro- 1,4-benzodioxin-6- ylamino)imidazo[1,2- a]pyrazin-2-yl] benzoate | 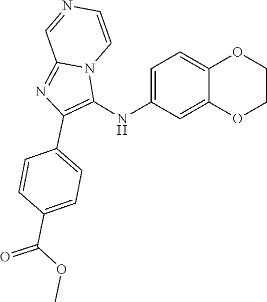 [SMILES: COC(=O)c1ccc(-c2nc3cnccn3c2Nc2ccc3c(c2)OCCO3)cc1] |
| P24872 | 27.16 | methyl 4-[3-(1,3-benzodioxol- 5-ylamino)imidazo [1,2-a]pyrazin- 2-yl]benzoate | 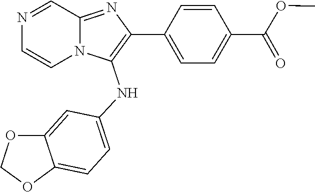 [SMILES: COC(=O)c1ccc(-c2nc3cnccn3c2Nc2ccc3c(c2)OCO3)cc1] |
| P28366 | 36.28 | N-(1,3-benzodioxol- 5-yl)-2-(4-fluoro- phenyl)imidazo[1,2- a]pyrazin-3-amine | 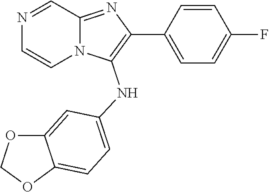 [SMILES: Fc1ccc(-c2nc3cnccn3c2Nc2ccc3c(c2)OCO3)cc1] |
| P28376 | 28.53 | methyl 4-[3-(4-methoxy- anilino)imidazo[1,2-a] pyrazin-2-yl]benzoate | 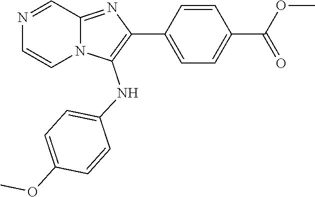 |
| P28734 | 41.03 | N-ethyl-3-[[2-(4- methoxyphenyl) imidazo[1,2-a]pyrazin- 3-yl]amino]benzamide | 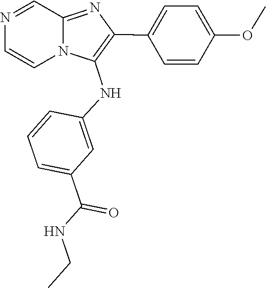 [SMILES: CCNC(=O)c1cccc(Nc2c(-c3ccc(OC)cc3)nc3cnccn23)c1] |
| P32333 | 35.78 | N-(2,6-dimethylphenyl)- 2-phenylimidazo [1,2-a]pyrazin-3- amine | 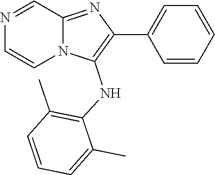 [SMILES: Cc1cccc(C)c1Nc1c(-c2ccccc2)nc2cnccn12] |
| P32343 | 12.93 | methyl 3-[[2-(3-bromophenyl) imidazo[1,2-a] pyrazin-3-yl]amino] benzoate | 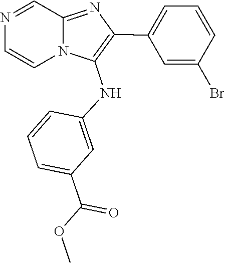 [SMILES: COC(=O)c1cccc(Nc2c(-c3cccc(Br)c3)nc3cnccn23)c1] |
| P36232 | 20.27 | methyl 4-[3-(2-ethyl-6- methylanilino)imidazo [1,2-a]pyrazin-2- yl]benzoate | 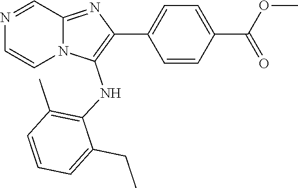 [SMILES: CCc1cccc(C)c1Nc1c(-c2ccc(C(=O)OC)cc2)nc2cnccn12] |
| P37287 | 21.01 | methyl 4-[3-(cyclopentyl- amino)imidazo[1,2- a]pyrazin-2-yl] benzoate | 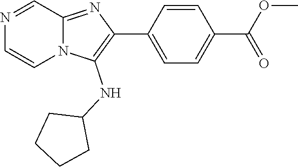 [SMILES: COC(=O)c1ccc(-c2nc3cnccn3c2NC2CCCC2)cc1] |
| P37288 | 13.18 | methyl 3-[[2-(3-hydroxy- phenyl)imidazo[1,2- a]pyrazin-3-yl] amino]benzoate | 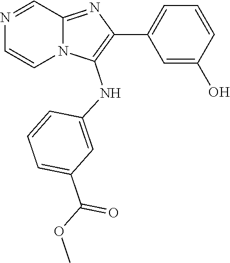 [SMILES: COC(=O)c1cccc(Nc2c(-c3cccc(O)c3)nc3cnccn23)c1] |
| P37297 | 22.29 | methyl 4-[3-(4-propan-2- ylanilino)imidazo[1,2- a]pyrazin-2-yl] benzoate | 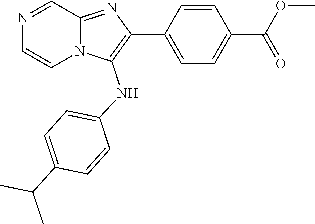 [SMILES: COC(=O)c1ccc(-c2nc3cnccn3c2Nc2ccc(C(C)C)cc2)cc1] |
| P37298 | 33.63 | 3-[[2-(4-methyl- sulfanylphenyl)imidazo [1,2-a]pyrazin-3- yl]amino]benzoic acid | 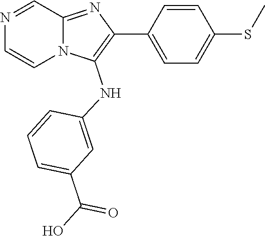 [SMILES: CSc1ccc(-c2nc3cnccn3c2Nc2cccc(C(=O)O)c2)cc1] |
| P37307 | 43.38 | methyl 4-[3-(2,6-dimethyl- anilino)imidazo[1,2- a]pyrazin-2-yl] benzoate | 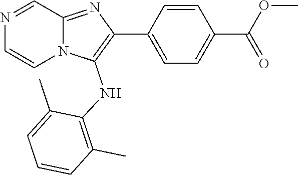 [SMILES: COC(=O)c1ccc(-c2nc3cnccn3c2Nc2c(C)cccc2C)cc1] |
| P37308 | 20.75 | 3-[[2-(4-methyl- phenyl)imidazo[1,2- a]pyrazin-3-yl]amino] benzoic acid | 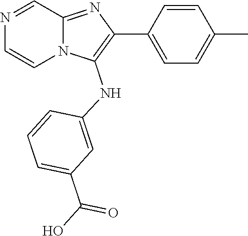 [SMILES: Cc1ccc(-c2nc3cnccn3c2Nc2cccc(C(=O)O)c2)cc1] |
| P37317 | 27.55 | methyl 4-[3-[3-(trifluoro- methyl)anilino]imidazo [1,2-a]pyrazin- 2-yl]benzoate | 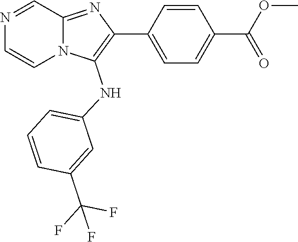 [SMILES: COC(=O)c1ccc(-c2nc3cnccn3c2Nc2cccc(C(F)(F)F)c2)cc1] |
| P37318 | 15.90 | 3-[[2-(2-hydroxy- phenyl)imidazo[1,2- a]pyrazin-3-yl]amino] benzoic acid | 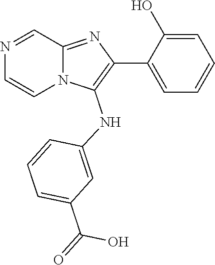 [SMILES: O=C(O)c1cccc(Nc2c(-c3ccccc3O)nc3cnccn23)c1] |
| P37327 | 48.52 | methyl 3-[[2-(4-hydroxy- phenyl)imidazo[1,2- a]pyrazin-3-yl]amino] benzoate | 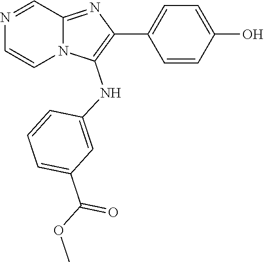 [SMILES: COC(=O)c1cccc(Nc2c(-c3ccc(O)cc3)nc3cnccn23)c1] |
| P37328 | 17.18 | 3-[[2-(4-nitrophenyl) imidazo[1,2-a] pyrazin-3-yl]amino] benzoic acid | 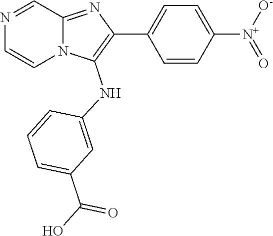 [SMILES: O=C(O)c1cccc(Nc2c(-c3ccc([N+](=O)[O-])cc3)nc3cnccn23)c1] |
| P37337 | 23.01 | methyl 3-[[2-(4-chlorophenyl) imidazo[1,2-a] pyrazin-3-yl]amino] benzoate | 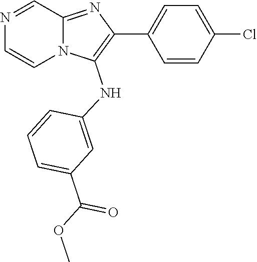 [SMILES: COC(=O)c1cccc(Nc2c(-c3ccc(Cl)cc3)nc3cnccn23)c1] |
| P37338 | 11.67 | 3-[[2-(3-hydroxy- phenyl)imidazo[1,2- a]pyrazin-3-yl]amino] benzoic acid | 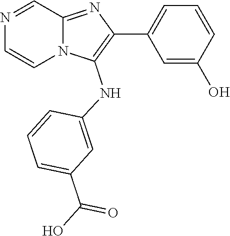 [SMILES: O=C(O)c1cccc(Nc2c(-c3cccc(O)c3)nc3cnccn23)c1] |
| P37347 | 24.96 | methyl 3-[[2-(2-hydroxy- phenyl)imidazo[1,2- a]pyrazin-3-yl]amino] benzoate | 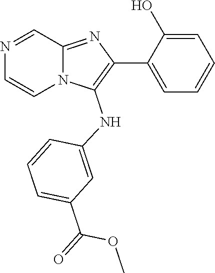 [SMILES: COC(=O)c1cccc(Nc2c(-c3ccccc3O)nc3cnccn23)c1] |
| P37348 | 16.02 | 2-(4-aminophenyl)- N-(2-ethyl-6-methyl- phenyl)imidazo[1,2- a]pyrazin-3-amine | 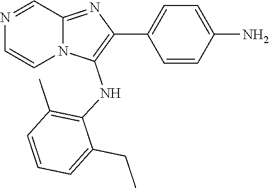 [SMILES: CCc1cccc(C)c1Nc1c(-c2ccc(N)cc2)nc2cnccn12] |
| P37356 | 3.18 | N-(4-chlorophenyl)- 2-(4-fluorophenyl) imidazo[1,2-a] pyrazin-3-amine | 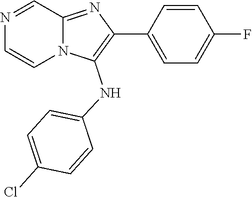 [SMILES: Fc1ccc(-c2nc3cnccn3c2Nc2ccc(Cl)cc2)cc1] |
| P37357 | 4.32 | methyl 3-[[2-(4-fluorophenyl) imidazo[1,2-a] pyrazin-3-yl]amino] benzoate | 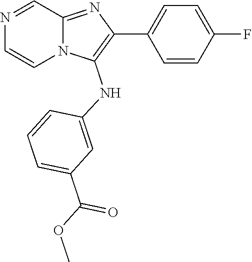 [SMILES: COC(=O)c1cccc(Nc2c(-c3ccc(F)cc3)nc3cnccn23)c1] |

### Embodiment 2: IC50 Determination of the Inhibitory of Imidazo [1, 2-a] Pyrazine Compounds on PLpro Activity of SARS-CoV-2

Using the same experimental principle as Embodiment 1. After adding different concentrations of imidazo [1, 2-a] pyrazine compounds to the reaction system, the changes in PLpro activity were reflected by the readings of a microplate reader, and the IC50 of small molecule compounds inhibiting PLpro was calculated. The specific steps are as follows:
2.1 The composition of the reaction buffer is DTT 10 mM, Tris 20 mM, NaCl 150 mM, 2% DMSO. The substrate RLRGG-AMC purchased from Beyotime was dissolved in DMSO to make a 10 mM stock solution. The PLpro of SARS-CoV-2 purchased from Sino Biological Inc. was dissolved to make a 125 ng/μL stock solution with nuclease-free water.
2.2 Preparation of substrate: adding 2 μL of substrate stock solution to 198 μL reaction buffer, and mixing well to obtain 100 μM of substrate working solution.
2.3 Preparation of PLpro: adding 10 μL of PLpro stock solution to 90 μL reaction buffer, and mixing well to obtain 12.5 ng/μL of PLpro working solution.
2.4 For the experimental groups, mixing 10 μL of PLpro working solution with 2 μL of imidazo [1, 2-a] pyrazine compounds (Final concentrations are 100 μM, 50 μM, 25 μM, 12.5 μM, 6.25 μM, 3.125 μM, 1.5625 μM, 0.7812 μM, 0.3906 μM, 0.1953 μM, 0.0976 μM, 0.0488 μM, 0.0244 μM); for the control group, not adding any compounds but mixing 10 μL of PLpro working solution with 2 μL of compound solvent (DMSO); then incubate at 37° C. for 10 minutes.
2.5 Quickly adding 10 μL of the substrate working solution prepared in 1.2 to the 2.4 system, then using a microplate reader to continuously read for 15 minutes under 37° C. incubation conditions, with a 30 second interval between each cycle, wherein the excitation wavelength is set to 380 nm and the emission wavelength is set to 460 nm.
2.6 Calculation of inhibition rate: taking time min as the X-axis and fluorescence value RFU as the Y-axis, fitting the linear region to obtain RFU/min as enzyme activity, then calculating the inhibition rate of the different concentrations of compounds to be tested on the PLpro of SARS-COV2 based on the difference in enzyme activity between the experimental groups and the control group. The calculation formula is as follows: inhibition rate=(1−enzyme activity of experimental group/enzyme activity of control group)×100%.
2.7 Calculation of IC50: calculating the IC50 of each derivative by using GraphPad Prism software, taking X-axis as the drug concentration and Y-axis as the enzyme inhibition rate.
   The inhibitory results of 21 imidazo [1, 2-a] pyrazine compounds on PLpro activity are shown in Table 2. Among them, the three compounds with the best inhibitory effect, P19453, P22445, and P22475, have IC50 values of 6.45 μM, 13.94 μM, and 16.57 μM, respectively.

TABLE 2
IC50 Results of Inhibition of Coronavirus PLpro Activity by
Imidazo [1,2-a] Pyrazine Compounds

| | Inhibition rate at | |
|---|---|---|
| Serial number | 50 μM (%) | IC50 (μM) |
| P22423 | 54.48 | 33.51 |
| P141 | 63.75 | 38.06 |
| P18224 | 54.57 | 38.35 |
| P19442 | 61.12 | 43.20 |
| P19443 | 53.01 | 31.10 |
| P19445 | 50.21 | 47.64 |
| P19446 | 65.76 | 30.26 |
| P19452 | 53.11 | 28.36 |
| P19453 | 65.52 | 6.454 |
| P19454 | 53.82 | 36.80 |
| P19455 | 53.10 | 26.58 |
| P19456 | 47.83 | 60.91 |
| P19462 | 64.99 | 26.00 |
| P22414 | 51.11 | 45.95 |
| P22415 | 63.34 | 49.72 |
| P22438 | 54.25 | 40.75 |
| P22445 | 62.30 | 13.94 |
| P22471 | 52.83 | 35.15 |
| P22475 | 60.09 | 16.57 |
| P22479 | 58.84 | 35.41 |

### Embodiment 3: SwissADME Evaluation of the Druggability of Compounds P19453, P22445, and P22475

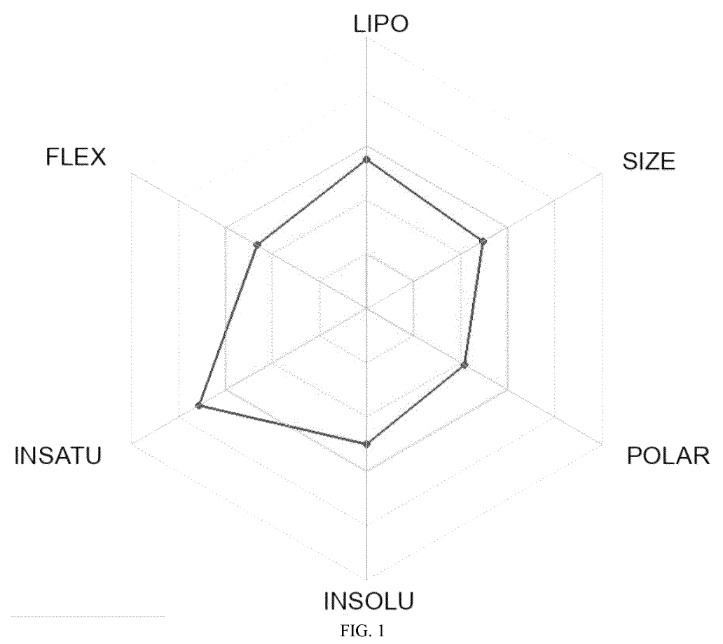

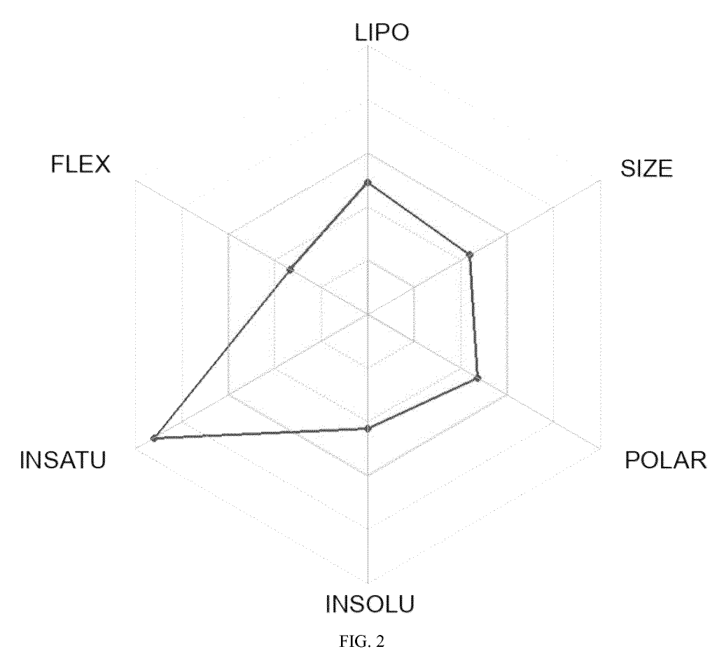

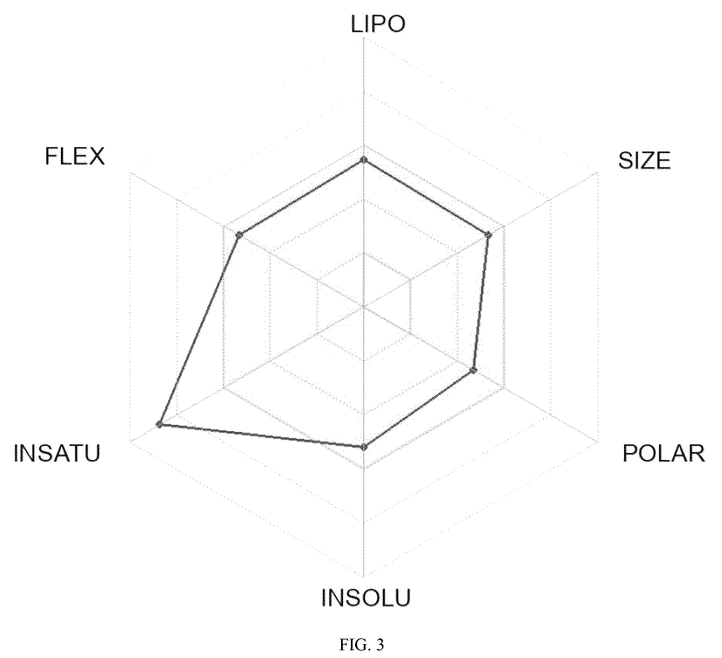

At SwissADME (http://www.swissadme.ch/), drawing the structures of compounds P19453, P22445, and P22475, and outputting their ADME properties for pharmacological evaluation. The evaluation results of SwissADME show that all three compounds have good druggability, as shown in FIG. 1 to FIG. 3.

### Embodiment 4: Detection of Cytotoxicity of Compounds P19453, P22445, and P22475

The cytotoxicity of P19453, P22445, and P22475 was detected through the CCK-8 assay, which was purchased from Shanghai Yisheng. The specific steps are as follows:
4.1 Cell plating: human bronchial epithelial cells (BEAS-2B cells) with logarithmic growth phase were seeded onto a 96 well plate (100 μl/well), with 3 wells in each group. The number of cells in each well was set to 5×10³, and cultured in a 5% CO₂ incubator at 37° C. for 12 hours.
4.2 Different concentrations of P19453, P22445, and P22475 were used for treatment, and the experimental group drugs were diluted with DMSO, and the drug concentration is set to 100 μM, 50 μM, 25 μM, 12.5 μM, 6.25 μM, 3.125 μM, and the dosage volume is 2 μL; adding 2 μL DMSO to the control group; then incubating in a 5% CO₂ incubator at 37° C.
4.3 After 24 hours, washing the cells twice with PBS and add 100 μL of pre-mixed CCK8 solution (culture medium: CCK8 working solution=9:1) to each well, avoiding the introduction of bubbles. Then culture the cells in the 5% CO₂ incubator at 37° C. for additional 2 hours.
4.4 Using a microplate reader to measure the absorbance D (λ) of each well at a wavelength of 450 nm, then analyzing the D (λ) values of the blank group, the control group, and the experimental groups to determine cell activity. The cell survival rate reflects the cytotoxicity of the drug. The groups are as follows: Experimental group (culture medium containing cells, CCK-8, drug to be tested) Control group (culture medium containing cells, CCK-8, without the drug under test) Blank group (culture medium without cells and drug to be tested, only containing CCK-8)

$$survival rate = \frac{{D ( λ )}_{experimental group} - {D ( λ )}_{blank group}}{{D ( λ )}_{control group} - {D ( λ )}_{blank group}} \times 100 \%$$

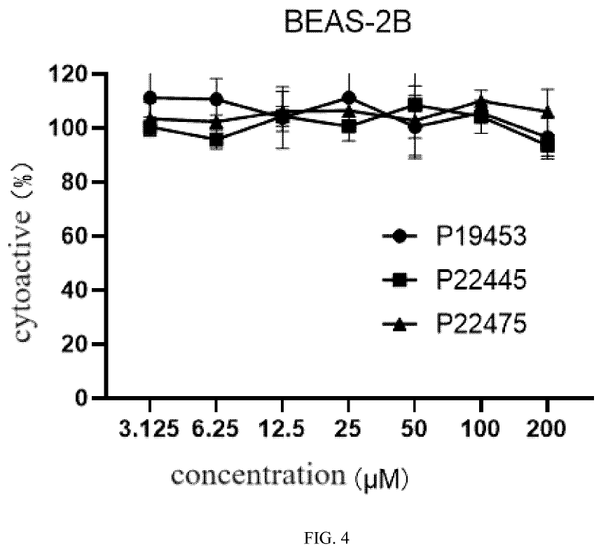

As shown in FIG. 4, the CCK8 results shows that the three compounds have no significant cytotoxicity to BEAS-2B cells within the tested concentration range (up to 200 μM).

1. An application of imidazo [1, 2-a] pyrazine compounds or their pharmaceutically acceptable salts in a preparation of anti-coronavirus drugs, characterized in that the imidazo [1, 2-a] pyrazine compounds are selected from the following compounds: 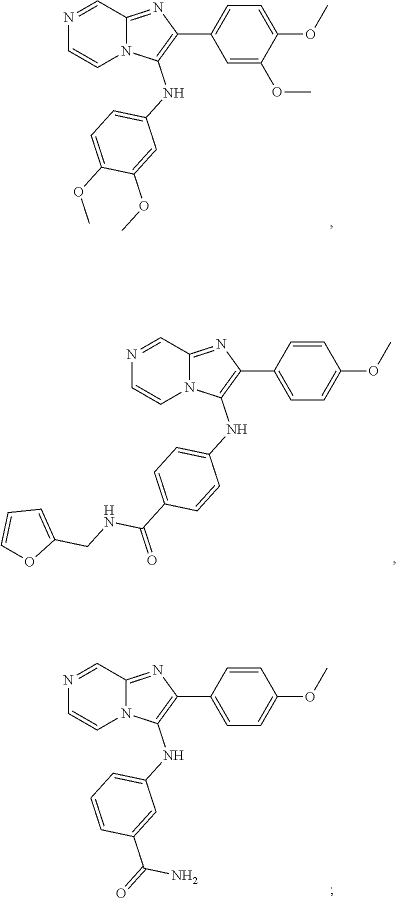 [SMILES: COc1ccc(Nc2c(-c3ccc(OC)c(OC)c3)nc3cnccn23)cc1OC ; COc1ccc(-c2nc3cnccn3c2Nc2ccc(C(=O)NCc3ccco3)cc2)cc1 ; COc1ccc(-c2nc3cnccn3c2Nc2cccc(C(N)=O)c2)cc1]
and
the coronavirus is SARS-CoV-2.
